(19) United States
(12) Patent Application Publication
(10) Pub. No.: US 2025/0208147 A1
(43) Pub. Date: Jun. 26, 2025

(54) BIOMARKERS FOR PREDICTING TYPE 2 DIABETES STATUS

(71) Applicant: Verily Life Sciences LLC, Dallas, TX (US)

(72) Inventors: Alessandra Breschi, Mountain View, CA (US); Katherine Drake, Boulder, CO (US)

(21) Appl. No.: 18/984,223

(22) Filed: Dec. 17, 2024

Related U.S. Application Data
(60) Provisional application No. 63/613,209, filed on Dec. 21, 2023.

(51) Int. Cl. G01N 33/68

(52) U.S. Cl. CPC G01N 33/6893; G01N 2800/042

(57) ABSTRACT

Provided herein is a method for determining a type 2 diabetes status in a subject, where the method includes obtaining a biological sample from the subject; determining the level of one or more proteins; and transforming the weighted sum of the levels of one or more proteins into a probability score, wherein an increase in the probability score indicates an increased likelihood of the type 2 diabetes status.

### CROSS-REFERENCE TO RELATED APPLICATIONS

This application claims priority to U.S. Provisional Application No. 63/613,209, filed Dec. 21, 2023, which is incorporated by reference herein in its entirety.

### BACKGROUND

Approximately 28 million people have been diagnosed with type 2 diabetes (T2D) in the US, with an additional 8.5 million people estimated to be undiagnosed. Current diagnostic criteria for diabetes and prediabetes involve measuring blood glucose levels and percentage of glycated hemoglobin (HbA1c) to determine whether levels are above the ‘normal references’ of 99 mg/dL and 5.7%, respectively. Common phenotypes of T2D include insulin resistance and hyperglycemia, but, in the entirety of its pathology, T2D is a complex disease often associated with other systemic alterations, such as obesity, lipid metabolism alterations, hypertension, chronic inflammation and endothelial damage. Because of the complexity of the disease, identification of additional markers could refine the stratification of diabetes phenotypes, and in turn, improve the personalization of follow-up and management.

### BRIEF SUMMARY

Various examples are described for determining a type 2 diabetes status using one or more biomarkers as described herein. One example method for determining type 2 diabetes status includes obtaining a biological sample from the subject; determining the level of one or more proteins; and transforming the weighted sum of the levels of one or more proteins into a probability score, wherein an increase in the probability score indicates an increased likelihood of a type-2 diabetes status in a subject.

These illustrative examples are mentioned not to limit or define the scope of this disclosure, but rather to provide examples to aid understanding thereof. Illustrative examples are discussed in the Detailed Description, which provides further description. Advantages offered by various examples may be further understood by examining this specification.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1D illustrate various aspects of differentially expressed proteins between diabetic and normoglycemic individuals. FIG. 1A is a volcano plot highlighting differentially expressed proteins. The x axis refers to the coefficient associated to the phenotype in the linear model for each protein. All samples are considered to compute the beta coefficient. FIG. 1B is a heatmap showing the Z-score of batch-corrected expression of DE proteins. Hierarchical clustering obtained with Euclidean distance and complete linkage. FIG. 1C illustrates a PPI network of co-expressed DE proteins after 2-core filtering. Communities are detected with Louvain algorithm. Nodes are colored based on selected Gene Ontology (GO) slim. The border of the node is colored based on the assigned community. Circles: upregulated; Triangles: downregulated. FIG. 1D shows expression of DE proteins between diabetic and normoglycemic subjects with normal weight. Expression is scaled with respect to the median expression in normoglycemic.

FIGS. 2A-2C illustrate aspects of transcriptomics analysis of differentially expressed (DE) proteins in liver, including expression levels and patterns of expression. FIG. 2A shows distribution of tissue-specific DE proteins at the transcriptional level in the GTEx dataset. FIG. 2B illustrates gene expression of DE proteins in scRNA-seq data from hepatocytes from different regions of the liver. The color of the dots is proportional to the scaled normalized gene expression values. The size of the dots is proportional to the proportion of cells of a given type expressing the gene. FIG. 2C is UMAP of selected DE proteins showing their zonation patterns of gene expression.

FIGS. 3A-3E illustrate aspects of clustering analyses of diabetic, prediabetic and normoglycemic participants based on clinical and proteomics data. FIGS. 3A, 3B are UMAP embeddings of selected proteins and clinical features after PCA, colored by diabetic status, FIG. 3A, and k-means cluster labels, FIG. 3B. FIG. 3C is a plot showing a silhouette profile of participants assigned to three clusters with k-means clustering on proteomics and clinical data projected in a 15-dimensional space with supervised PCA. FIG. 3D is a table of the number of participants assigned to each cluster, by diabetic status, colored by relative distribution across diabetic status within each cluster. FIG. 3E is a heatmap of normalized expression of DE proteins between normoglycemic and diabetic participants and between participants assigned to the normoglycemic-like or the diabetic-like clusters. DE proteins are filtered at FDR<=0.01 and absolute coefficient>=0.15 for visualization. Participants are sorted by cluster, diabetic status, body mass index (BMI) diagnosis and age, in this order.

FIGS. 4A-4C are plots illustrating differences in external features (metabolic, physical, and echocardiogram) between clusters. FDR-adjusted p-value comparing features between normoglycemic-like and diabetic-like clusters for each observed phenotype: A: 0.01-0.05, *: 0.001-0.01, **: 0.0001-0.001, ***: <0.0001 (Mann-Whitney test). Because of sample size, female and male participants are pooled together for testing. Features are distributed into three main groups based on feature type: metabolic features (FIG. 4A), physical performance features (FIG. 4B) and derived features from echocardiogram (FIG. 4C).

FIGS. 5A-5D illustrate various aspects of machine learning predictions of type 2 diabetes. FIG. 5A shows plots of performance metrics of a ridge logistic regression model predicting diabetic status. Performance metrics are computed for each repeated CV (repeated) or for each repeated nested CV (nested) iteration. Differences between performance metrics of models trained with different datasets are tested with one-sided Mann-Whitney test, comparing the concatenated datasets to clinical and proteomics only, correcting for multiple testing. Adjusted p-value legend: A: 0.01-0.05, *: 0.001-0.01, **: 0.0001-0.001, ***: <0.0001. FIG. 5B is a confusion matrix of diabetes prediction in prediabetics and cluster labels. FIG. 5C shows number of samples predicted diabetic where feature has the highest SHAP value. Red: protein is DE and overexpressed in diabetics, Blue: protein is DE and underexpressed in diabetics. FIG. 5D illustrates features contributing to diabetes predictions in prediabetic participants, divided by cluster assignment. Ten prediabetic participants predicted normoglycemic are also shown as control. Circle size is proportional to the positive SHAP value for each participant and feature, circle color is proportional to the feature value scaled across all participants with proteomics data.

FIGS. 6A and 6B illustrate aspects of the study cohort showing cohort distribution and cohort selection process. FIG. 6A is a graph of distribution of cohorts at different study points. FIG. 6B is a flow chart showing participant flow through the cohort selection process.

FIGS. 7A-7C are plots illustrating various aspects of proteomics quality assessment. FIG. 7A show graphs of the distribution of protein-level coefficient of variation between the two technical replicates for each sample (x-axis). Each box represents a batch. Horizontal lines at 0.05 and 0.1 coefficients of variation are plotted for reference. FIG. 7B is a graph of number of undetected proteins per sample. FIG. 7C is a graph showing relationship between C-Reactive Protein expression quantified by LC/MS (x-axis) and by standard clinical blood test (y-axis), colored by batch. LC/MS expression values are not corrected for batch effect.

FIG. 8 is a plot showing stability of DE proteins across 10 random subsets of 90% of the samples. For each random subset, 90% of the samples were selected with resampling allowed across subsets. Differential expression analysis was performed (see Methods) and a Benjamini-Hochberg adjusted p-value was calculated for each protein in each subset. Proteins were considered significant if adjusted p<0.05 across all random subsets.

FIG. 9 is a GO enrichment heatmap, showing Biological Processes and Cellular Component.

FIG. 10 is a heatmap of pairwise Pearson's correlation coefficients between clinical features, including results from blood and urine analysis and vitals at study start. Features were selected for clustering and machine learning based on a combination of data availability (at least 50% of the participants), correlation between features (Pearson r>0.8), and only including features from blood tests and vitals.

FIG. 11 illustrates the results of a single-feature analysis of association between each clinical feature and diabetes status at study start, for participants having proteomics data. A linear model was built for each feature where the value of the feature is modeled as a function of diabetes status, age, sex, race, smoking status, comorbidities, medications for hypertension and statins. The coefficients associated to diabetes status are shown with their 95% confidence interval. Significance is assessed at 5% q-value after adjusting for multiple testing with Benjamini-Hochberg correction. Feature values are scaled so coefficients can be compared. Features selected for the clustering and machine learning are marked with an asterisk.

FIGS. 12A-12C are plots showing the evaluation of different clustering algorithms. FIG. 12A is a plot showing proportion of variance explained by an increasing number of principal components from clinical and proteomic features selected for clustering. FIG. 12B illustrates plots of 5 different clustering metrics (rows: ssw, sil, ch, ami, db) evaluated for varying numbers of clusters (k), principal components used as input (columns) and clustering methods (colored lines). FIG. 12C are additional plots where for k=3 for each clustering method (rows) and each number of principal components used as input (columns), number of participants with each diagnosis in each cluster.

FIGS. 13A-13C and plots showing the differences between of external features between clusters. FDR-adjusted p-value comparing features between normoglycemic-like and diabetic-like clusters for each observed phenotype: \hat{ }: 0.01-0.05, *: 0.001-0.01, **: 0.0001-0.001, ***: <0.0001 (Mann-Whitney test). Because of sample size, female and male participants are pooled together for testing. Features are distributed into three main groups based on feature type: metabolic features (FIG. 13A), physical performance features (FIG. 13B) and derived features from echocardiogram (FIG. 13C).

FIGS. 14A-14C are diagrams illustrating aspects of models utilized according to various aspects of the present disclosure. FIG. 14A is a diagram showing pre-processing and modeling steps for each ML model. Where feature selection was performed, the number of features selected was tuned as a hyperparameter within the cross-validation framework. FIG. 14B shows cross-validation framework for model selection, which was used to build a model for interpretation. FIG. 14AC shows Cross-validation framework for model performance evaluation.

### DETAILED DESCRIPTION

Understanding diabetes at the molecular level can help refine diagnostic approaches and personalized treatment efforts. As described herein, proteomic data was generated from plasma collected from participants in a large longitudinal cohort (evaluable cohort from the Project Baseline Health Study, n=732), and integrated those data with information from their medical history and laboratory tests to determine diabetes status. Biomarker proteins were identified that were associated with diabetes status. Specifically, 87 differentially expressed proteins in people with diabetes, 71 of which showed higher expression. This proteomic profile was integrated with clinical data into a logistic regression model that could predict diabetes status with over 85% balanced accuracy (calculated as average recall, which is the average over recall computed for the positive label (with T2D) and the negative label (normoglycemic)). The approach described herein indicates that proteomic data can enhance diabetes phenotyping, which helps identify people with diabetes to target them with personalized treatments or interventions.

Provided herein is a multi-protein signature that can help classify type 2 diabetes disease status in a subject. Information relating to the multi-protein signature (e.g., expression levels of proteins of the multi-protein signature taken together that can be transformed and analyzed) can also inform treatment status of the subject. The signature includes the levels of multiple proteins in a biological sample of a subject. The abundances of these proteins are then fed into a statistical model to assign a probability and classification of type 2 diabetes status.

The advantages of identifying patients currently with type 2 diabetes allows patients and health care providers to implement treatment measures, and/or change treatment plans to minimize or manage one or more symptoms of the disease. The multi-protein signature or panel may also be combined with additional clinical data such as medical history, treatment history, demographic information, time to last relapse, or other clinical lab markers.

Provided herein is a method for determining a type 2 disease status in a subject. The methods can include obtaining a biological sample from the subject; determining the level of one or more proteins; and transforming the levels of one or more proteins into a probability score, wherein an increase in the probability score indicates an increased likelihood of type 2 diabetes. Levels of one or more proteins from the following groups listed below can be assayed according to the present disclosure.

The one or more proteins can be electron transfer flavoprotein dehydrogenase (ETFDH), albumin (ALB), keratin 81, 83, 86 (KRT81; KRT83; KRT86), paraoxonase 1 (PON1), paraoxonase 3 (PON3), adiponectin, C1Q and collagen domain containing (ADIPOQ), sex hormone binding globulin (SHBG), apolipoprotein D (APOD), apolipoprotein A1 (APOA1), apolipoprotein M (APOM), cholesteryl ester transfer protein (CETP), cartilage acidic protein 1 (CRTAC1), GLI pathogenesis-related 2 (GLIPR2), cadherin 13 (CDH13), C-type lectin domain family 3 member B (CLEC3B), gelsolin (GSN), complement C7 (C7), complement C7; fibroblast activation protein alpha (C7; FAP), collectin subfamily member 10 (COLEC10), collectin subfamily member 11 (COLEC11), heat shock protein family A (Hsp70) member 5 (HSPA5), heat shock protein family A (Hsp70) member 5; heat shock protein family A (Hsp70) member 8 (HSPA5; HSPA8), fc gamma binding protein (FCGBP), colony stimulating factor 1 receptor (CSF1R), quiescin sulfhydryl oxidase 1 (QSOX1), fumarylacetoacetate hydrolase (FAH), galectin 3 binding protein (LGALS3BP), polymeric immunoglobulin receptor (PIGR), apolipoprotein A5 (APOA5), cathepsin D (CTSD), serpin family D member 1 (SERPIND1), haptoglobin (HP), haptoglobin; haptoglobin-related protein (HP;HPR), serum amyloid A1 (SAA1), S100 calcium binding protein A8 (S100A8), S100 calcium binding protein A9 (S100A9), procollagen C-endopeptidase enhancer (PCOLCE), fibrinogen gamma chain (FGG), fibrinogen alpha chain (FGA), fibrinogen beta chain (FGB), complement C8 alpha chain (C8A), complement C8 gamma chain (C8G), complement C6 (C6), complement C9 (C9), inter-alpha-trypsin inhibitor heavy chain 3 (ITIH3), gamma-glutamyl hydrolase (GGH), C-reactive protein (CRP), lipopolysaccharide binding protein (LBP), complement C2 (C2), mannosidase alpha class 1A member 1 (MAN1A1), apolipoprotein C4 (APOC4), apolipoprotein C2 (APOC2), apolipoprotein C3 (APOC3), apolipoprotein A4 (APOA4), apolipoprotein H (APOH), alpha-1-microglobulin/bikunin precursor (AMBP), serpin family F member 1 (SERPINF1), complement Clq B chain (C1QB), complement Clq C chain (C1QC), complement CIr subcomponent like (ClRL), complement Clr (C1R), complement Cls (CIS), serpin family A member 10 (SERPINA10), coagulation factor XI (F11), protein C, inactivator of coagulation factors Va and VIIIa (PROC), serpin family F member 2 (SERPINF2), complement factor properdin (CFP), biotinidase (BTD), butyrylcholinesterase (BCHE), afamin (AFM), attractin (ATRN), complement factor H; complement factor H related 2 (CFH;CFHR2), complement C3 (C3), complement factor H (CFH), complement factor B (CFB), complement factor I (CFI), kininogen 1 (KNG1), vitronectin (VTN), complement C5 (C5), hemopexin (HPX), coagulation factor X (F10), orosomucoid 2 (ORM2), complement component 4 binding protein alpha (C4BPA), protein S (PROS1), proteoglycan 4 (PRG4), amyloid P component, serum (APCS), and coagulation factor IX (F9), or any combination thereof.

Optionally, the one or more proteins can be CDH13, CETP, CLEC3B, CRTAC1, GSN, MMP2, SHBG, C3, CFB, VTN, or any combination thereof.

Optionally, the one or more proteins can be AMBP, ALB, APOA1, HP, SAA1, APOC3, HPX, APOH, VTN, ORM2, APCS, APOA5, FGG, FGB, FGA, CRP, ITIH3, KNG1, SERPINF2, C3, C6, CFH, C5, C8A, AFM, C4BPA, C9, LBP, CFI, PON1, F11, PROC, F9, APOM, CFB, C2, SERPIND1, SERPINA10, F10, PRG4, BCHE, PON3, APOA4, SHBG, APOC2, COLEC10, APOC4, C8G, PIGR, COLEC11, or any combination thereof.

Optionally, the one or more proteins can be APOA1, APOM, SAA1, CFI, C5, F11, or any combination thereof.

Optionally, the one or more proteins can be SHBG, FAH, LGALS3BP, PIGR, GGH, C1RL, MAN1A1, CRP, LBP, C9, FGA, FGG, SAA1, S100A8, S100A9, SERPIND1, HP, HP;HPR, APOC4, ORM2, CFH;CFHR2, C3, CFH, CFB, CFI, PRG4, APCS, F9, or any combination thereof.

Optionally, the one or more proteins can be ITIH3, PROS1, ATRN, C3, C2, BTD, FCGBP, PIGR, C7, SAA1, APOA4, VTN, APOC2, APOA5, C1QC, APOC3, QSOX1, C8A, CFI, GSN, SHBG, APOM, CETP, APOD, ADIPOQ, or any combination thereof.

Optionally, the one or more proteins can be ITIH3, PROS1, ATRN, C3, C2, BTD, FCGBP, PIGR, C7, SAA1, APOA4, VTN, APOC2, APOA5, C1QC, APOC3, QSOX1, C8A, CFI, or any combination thereof.

Optionally, the one or more proteins can be GSN, SHBG, APOM, CETP, APOD, DIPOQ, or any combination thereof.

Optionally, the one or more proteins can be SHBG, APOD, C3, VTN, C2, GSN, CFB, CFH, APOA1, CFH;CFHR2, CFI, QSOX1, ADIPOQ, HSPA5;HSPA8, C4BPA, ATRN, PON3, CETP, PIGR, SERPIND1, PROS1, FGA, C7, APOC4, FGB, FGG, C1RL, BTD, LGALS3BP, F9. HPX, CDH13, GGH, CTSD, SERPINF1, CLEC3B, HP;HPR, NKG1, CRP, CRTAC1, COLEC10, LBP, C5, PCOLCE, AFM, C1QB, KRT81;KRT83;KRT86, APOC3, ETFDH, C6, BCHE, APOM, HP, PRG4, C8G, SERPINA10, APOC2, SERPINF2, ALB, APCS, COLEC11, FCGBP, F11, or any combination thereof.

Optionally, the one or more proteins can be CFP, CFB, CFH, C3, C9, C8G, C8A, C5, C7, S100A9, S100A8, LBP, FGB, FGA, APCS, CSF1R, C6, CFI, C4BPA, C2, CIS, C1RL, C1R, C1QB, C1QC, or any combination thereof.

Optionally, the one or more proteins can be PROS1, FGB, F9, C7, C5CFI, VTN, CFB, C4BPA, CETP, APOD, APOC1, APOM, APOA1, APOH, APOC4, APOC3, APOC2, APOA4, LCAT, or any combination thereof.

The methods can include detecting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, or 87 of the proteins. Thus, the method can include detecting 2 or more of the proteins. Optionally, the method includes detecting all of the proteins listed above.

Optionally, the one or more proteins can consist or consist essentially of any of the proteins noted above, for example, where “the one or more proteins can be PROS1, FGB, F9, C7, C5CFI, VTN, CFB, C4BPA, CETP, APOD, APOC1, APOM, APOA1, APOH, APOC4, APOC3, APOC2, APOA4, LCAT, or any combination thereof,” it would be understood that the one or more proteins can comprise one or more of the proteins (or any combination thereof), consist of one or more of the proteins (or any combination thereof), or consist essentially of one or more of the proteins (or any combination thereof) noted in a grouping.

In the provided methods, transforming the levels of one or more proteins into a probability score can include applying a statistical model (e.g., linear models) to the determined levels of the one or more proteins to assign a probability score for the subject. The levels of one or more proteins can be normalized before applying a statistical model. Normalization or transformation may include adjusting the expression level of the proteins in relation to the average protein levels in a sample. The statistical model can be a classification (i.e., logistic regression). The statistical model can be trained using datasets and databases as described herein, or similar data sets, in order to determine a probability against which calculated probability scores can be compared to. Training datasets can be restricted to those that only comprise protein levels of proteins described herein. Optionally, the probability score calculation is based on the continuous change of protein abundances, instead of a binary threshold of statistically significant vs. non-significant increase/decrease in abundance.

As used herein, “probability score” refers to the value assigned to a subject based on analysis of the abundances of one or more proteins, e.g., the proteins of listed above, and converting the abundances through transformation and statistical analyses to the value. The probability score is used to define a subject's type 2 diabetes status. The probability score can be calculated using the weighted sum of the proteins analyzed. The probability score can also be calculated using the protein abundances or weighted sum of the proteins as well as other patient information or other clinical features. Such information can include, for example, demographic factors, medical history of the subject, or any combination thereof. The probability score can be calculated using methods known in the art.

The method can include analyzing other information to assign a probability score to the subject. For example, the method can further comprise reviewing clinical features (i.e., demographic factors and clinical features related to past or present medical history of the subject) to assign the probability score to the subject.

Clinical features of a subject can be integrated with proteomics analysis to generate a probability score. Clinical features can include, for example, sex at birth, racial identity, age, one or more respiratory rate measurements, one or more triglyceride measurements, one or more waist circumference measurements, one or more glycated hemoglobin (HbA1c) measurements, one or more blood glucose measurements, one or more fasting blood glucose measurements, hypercholesterolemia status, hypertension status, one or more oral glucose tolerance test (OGTT) results, one or more total cholesterol measurements, one or more low-density lipoprotein (LDL) measurements, one or more high-density lipoprotein (HDL) measurements, one or more weight measurements, one or more body mass index (BMI) calculations, one or more blood pressure (BP) measurements, one or more pulse rate measurements, one or more average step count measurements, one or more methylation age measurements, one or more echocardiogram images, one or more ventricular mass measurements, one or more ventricular septal measurements, one or more mitral valve blood flow measurements, or any combination of any thereof.

Optionally, the clinical features can include age, sex, comorbidity status, hypertension medication status, statin status, diabetes medication status, or any combination of any thereof.

Optionally, the clinical features can include sex at birth, one or more HbA1c % measurements, one or more random glucose measurements, one or more BMI measurements, one or more systolic BP measurements, age, biological age, one or more pulse measurements, one or more 6 minute challenge measurements, one or more 10 meter challenge (fast pace) measurements, one or more 10 meter challenge (comfort pace) measurements, one or more 30 second stair stand challenge measurements, average daily step counts, one or more left ventricular inter ventricular septal thickness measurements, one or more left ventricular mass measurements, one or more mitral valve E/A ratio measurements, one or more mitral valve E/A ratio peak measurements, one or more septal peak e′ velocity measurements, or any combination of any thereof.

Optionally, the clinical features can include age, race, one or more absolute basophil measurements, one or more BMI measurements, one or more systolic BP measurements, one or more mean corpuscular volume (MCV) measurements, one or more hemoglobin measurements, one or more total cholesterol measurements, one or more magnesium measurements, one or more triglyceride measurements, one or more chloride measurements, one or more HDL cholesterol direct measurements, one or more platelet count measurements, one or more absolute lymphocyte measurements, or any combination of any thereof.

Optionally, the clinical features can include one or more BMI measurements, age, one or more pulse measurements, one or more systolic BP measurements, one or more aggregated complement protein measurements, one or more aggregated blood coagulation protein measurements, one or more LDL measurements, one or more triglyceride measurements, one or more absolute basophil measurements, one or more platelet count measurements, one or more MCV measurements, one or more HDL measurements, one or more total cholesterol measurements, one or more magnesium measurements, one or more chloride measurements, or any combination of any thereof.

Optionally, the clinical features can include sex, age, race, smoking status, comorbidity status, statin usage status, hypertension medication usage status, or any combination of any thereof.

Optionally, the clinical features can include mean corpuscular hemoglobin concentration (MCHC), mean corpuscular hemoglobin (MCH), MCV, bilirubin direct, bilirubin total, HDL direct, vitamin D, carbon dioxide, magnesium, reaction pH, eosinophils, eosinophils absolute, basophils, basophils absolute, lactic dehydrogenase, alanine aminotransferase (ALAT), aspartate aminotransferase (ASAT), albumin urine, albumin/creatine ratio, enzymatic creatinine serum, urea nitrogen, chloride, sodium, potassium, t-4 (thyroxine) free, phosphorus (inorganic), mean platelet volume (MPV), thyroid stimulating hormone, red cell count, hematocrit, hemoglobin, total cholesterol, LDL, total serum protein, albumin, calcium, lymphocytes, volgens Modification of Diet in Renal Disease (MDRD), glomerular filtration rate (GFR), absolute lymphocytes, platelet count, creatinine random ur, specific gravity, reticulocytes %, reticulocytes absolute, glucose, HbA1c, bmi, waist circumference, alkaline phosphatase, gamma-glutamyl transferase, respiratory rate, c-reactive protein (CRP) high sensitivity, pulse, diastolic (BP), systolic (BP), triglycerides, uric acid, neutrophil segmentation, total neutrophils, white cell count, absolute neutrophils, total neutrophils absolute, or any combination of any thereof.

Optionally, the clinical features can include BMI, triglycerides, pulse, absolute lymphocytes, absolute basophiles, platelet count, crp high sensitivity, gfr mdrd, alat (sgpt), red cell count, absolute neutrophils, absolute monocytes, absolute reticulocytes, absolute eosinophils, calcium, systolic bp, respiratory rate, potassium, asat (sgot), diastolic bp, eric acid, protein total serum, thyroid stimulating hormone, mpv, creatinine enz serum, mchc, total cholesterol, albumin, hemoglobin, vitamin D, sodium, chloride, magnesium, hdl cholesterol direct, mcv, or any combination of any thereof.

Optionally, one or more clinical features are not blood cell percentages, waist circumference, calculated LDL, or hematocrit.

Optionally, the clinical features can include one or more blood glucose measurements, one or more glycated hemoglobin (HbA1c) measurements, or both.

Optionally, the clinical features are not HbA1c or blood glucose measurements.

Optionally, the clinical features are one or more HbA1c measurements, one or more BMI measurements, one or more systolic BP measurements, one or more glucose measurements, one or more physical performance measurements, or any combination of any thereof.

Optionally, the clinical features are one or more left ventricular size measurements, one or more left ventricular mass measurements, one or more left ventricular septal thickness measurements, one or more mitral valve blood flow measurements, one or more mitral valve E/A ratio measurements, one or more septal peak e′ velocity measurements, one or more mitral valve E/e′ ratio measurements, one or more mitral valve E/A ratio peak measurements, or any combination of any thereof.

Optionally, the clinical features are one or more BMI measurements, age, one or more blood pressure measurements, one or more triglyceride measurements, one or more magnesium measurements, one or more chloride measurements, or any combination of any thereof.

Optionally, the clinical features can consist or consist essentially of any of the groupings of clinical features noted above, for example, where “the clinical features can include sex, age, race, smoking status, comorbidity status, statin usage status, hypertension medication usage status, or any combination of any thereof,” it would be understood that the clinical features can comprise one or more of the features (or any combination thereof), consist of one or more of the clinical features (or any combination thereof), or consist essentially of one or more of the clinical features (or any combination thereof) noted in a grouping.

The probability score can be used to determine the type 2 diabetes status (i.e., indicate a likelihood of type 2 diabetes status). The cutoff for type 2 diabetes status may be determined by combining statistical modeling and clinical domain knowledge. Without intending to be bound by any particular theory, patients with scores above the median across all patients can be deemed to be having type 2 diabetes.

The methods may include changing, adding or modifying one or more therapeutic treatments for the subject based on the probability score determined. For example, in the provided methods, if the subject is classified as having a type 2 diabetes status, the method may further comprise giving one or more therapeutic treatments for the subject. By way of another example, if the probability score indicates a current type 2 diabetes status, the method can include changing, adding or modifying one or more existing therapeutic treatments for the subject. Thus, the method can include adding one or more additional therapeutic treatments if a type 2 diabetes status is determined. The method may also optionally include adjusting the dosage of medication for a subject that is currently taking a medication that is determined to have a likelihood of type 2 diabetes status according to methods of the present disclosure. Therapeutic treatments as described herein can further comprise lifestyle interventions, for example, diet and weight loss interventions.

In the methods set forth herein, the biological sample may be derived from a subject and includes, but is not limited to, any cell, tissue or biological fluid. For example, the sample can be a tissue biopsy, whole blood or components thereof (e.g., plasma, serum, etc.), bone marrow, urine, saliva, tissue infiltrate, stool, saliva, tears, urine, one or more facial swabs and the like. Optionally, the samples is whole blood or urine. The biological sample may not be urine in some examples. The biological fluid may be a cell culture medium or supernatant of cultured cells from a subject.

Proteins can be detected using methods standard in the art for detecting and/or quantitating proteins. For example, proteins can be detected by densitometry, absorbance assays, fluorometric assays, Western blotting, ELISA, ELISPOT, immunoprecipitation, immunofluorescence (e.g., FACS), immunohistochemistry, and sequencing. Optionally, the level of the one or more proteins is determined using an assay selected from the group consisting of an enzyme-linked immunosorbent assay, a flow cytometry analysis, a dot blot assay, a Western blot assay, sequencing, liquid chromatography mass spectrometry (LCMS), orbitrap mass spectrometry, and an immunohistochemical localization assay.

Immunodetection methods are used for detecting, binding, purifying, removing and quantifying various molecules, including the disclosed proteins. Further, antibodies and ligands to the disclosed proteins can be detected. For example, the disclosed proteins are employed to detect antibodies having reactivity thereto. The steps of various useful immunodetection methods have been described in the scientific literature, such as, e.g., Maggio et al., Enzyme-Immunoassay (1987) and Nakamura et al., Enzyme Immunoassays: Heterogeneous and Homogeneous Systems, Handbook of Experimental Immunology, Vol. 1: Immunochemistry, 27.1-27.20 (1986), each of which is incorporated herein by reference in its entirety and specifically for its teaching regarding immunodetection methods. Immunoassays, in their most simple and direct sense, are binding assays involving binding between antibodies and antigen. Many types and formats of immunoassays are known, and all are suitable for detecting the disclosed biomarkers. Examples of immunoassays are enzyme linked immunosorbent assays (ELISAs), radioimmunoassays (RIA), radioimmune precipitation assays (RIPA), immunobead capture assays, Western blotting, dot blotting, gel-shift assays, flow cytometry, protein arrays, multiplexed bead arrays, magnetic capture, in vivo imaging, fluorescence resonance energy transfer (FRET), and fluorescence recovery/localization after photobleaching (FRAP/FLAP).

Based on the probability score, the method can include prescribing or administering a therapeutic agent to the subject. In the herein provided methods, the subject can already be receiving one or more therapeutic agents and the method can include changing the dose or therapeutic agent given to the subject. The dose of the therapeutic agent can be changed (i.e., modified) to increase or decrease the dose or amount of the therapeutic agent given to the subject. Optionally, the subject can already be receiving one or more therapeutic agents and the method can include administering to the subject an additional therapeutic agent.

The therapeutic agent that is being administered to the subject or the “additional” therapeutic agent that is administered can be an antibody, an anti-inflammatory agent, an immunomodulating agent, a steroid, plasmapheresis, gammaglobulin or a combination thereof. Optionally, the therapeutic agent is an agent used for treating type 2 diabetes or an agent similar to those used for treating type 2 or type 1 diabetes. Optionally, the therapeutic agent is metformin, pioglitazone, glimepiride, exenatide, canagliflozin, empagliflozin, dapagliflozin, dulaglutide, glimepiride, glibenclamide, glipizide, glucagon, chlorpropamide, glyburide, sitagliptin, saxagliptin, linagliptin, alogliptin, semaglutide, liraglutide, insulin, or an agent similar to these agents or any combination of these agents.

In the provided methods, administering therapeutic agents or altering therapeutic treatments given to the subject can reduce one or more symptoms of type 2 diabetes selected from the group consisting of hyperglycemia, fatigue, blurry vision, weight loss, excessive urination, excessive and persistent thirst, slow healing cuts or wounds, or other symptoms. The reduction can be a reduction of 1%, 5%, 10%, 15%, 20%, 30%, 40%, 50%, 60%, 70%, 80%, 90%, 95%, 99% or 100% as compared to a control subject. A control subject or value refers to a subject that serves as a reference, usually a known reference, for comparison. A control can also represent an average value gathered from a population of similar individuals, e.g., type 2 diabetic patients, with a similar medical background, same age, weight, and the like, but without therapeutic agents administered. A control value can also be obtained from the same individual, e.g., from an earlier-obtained sample, prior to disease, or prior to treatment.

Therapeutic agents can be administered to subjects using a pharmaceutical composition. Suitable formulations for use in a pharmaceutical composition can be found in Remington: The Science and Practice of Pharmacy 23rd edition, Adejare et al, editors, Elsevier (2020).

A pharmaceutically acceptable carrier can be a solid, semi-solid, or liquid material that can act as a vehicle, carrier or medium for the therapeutic agent. Thus, compositions containing one or more of the provided agents can be in the form of injections, tablets, pills, powders, lozenges, sachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders. Examples of the pharmaceutically-acceptable carriers include, but are not limited to, sterile water, saline, buffered solutions like Ringer's solution, and dextrose solution. The pH of the solution is generally from about 5 to about 8 or from about 7 to about 7.5.

Pharmaceutical compositions containing one or more therapeutic agents may be formulated for infusion. For intravenous infusions, there are two types of fluids that are commonly used, crystalloids and colloids. Crystalloids are aqueous solutions of mineral salts or other water-soluble molecules. Colloids contain larger insoluble molecules, such as gelatin; blood itself is a colloid. The most commonly used crystalloid fluid is normal saline, a solution of sodium chloride at 0.9% concentration, which is close to the concentration in the blood (isotonic). Ringer's lactate or Ringer's acetate is another isotonic solution often used for large-volume fluid replacement. A solution of 5% dextrose in water, sometimes called D5W, is often used instead if the patient is at risk for having low blood sugar or high sodium.

Combinations of different therapeutic agents may be administered either concomitantly (e.g., as an admixture), separately but simultaneously (e.g., via separate injection sites into the same subject), or sequentially (e.g., one of the components is given first followed by the second). Thus, the term combination is used to refer to either concomitant, simultaneous, or sequential administration of two or more agents.

According to the methods taught herein, the subject is administered an effective amount of the agent. The terms effective amount and effective dosage are used interchangeably. The term effective amount is defined as any amount necessary to produce a desired physiologic response. Effective amounts and schedules for administering the agent can be determined empirically, and making such determinations is within the skill in the art. The dosage ranges for administration are those large enough to produce the desired effect in which one or more symptoms of the disease or disorder are affected (e.g., reduced or delayed). The dosage should not be so large as to cause substantial adverse side effects, such as unwanted cross-reactions, anaphylactic reactions, and the like. Generally, the dosage will vary with the activity of the specific compound employed, the metabolic stability and length of action of that compound, the species, age, body weight, general health, sex and diet of the subject, the mode and time of administration, rate of excretion, drug combination, and severity of the particular condition and can be determined by one of skill in the art. The dosage can be adjusted by the individual physician in the event of any contraindications. Dosages can vary, and can be administered in one or more dose administrations daily, for one or several days. Guidance can be found in the literature for appropriate dosages for given classes of pharmaceutical products.

Any appropriate route of administration can be employed, for example, parenteral, intravenous, subcutaneous, intramuscular, intraventricular, intracorporeal, intraperitoneal, rectal, or oral administration. Administration can be systemic or local. Pharmaceutical compositions can be delivered locally to the area in need of treatment, for example by topical application or local injection. Multiple administrations and/or dosages can also be used. Effective doses for any of the administration methods described herein can be extrapolated from dose-response curves derived from in vitro or animal model test systems.

As used throughout, the term “subject” refers to an individual. Preferably, the subject is a mammal such as a primate, and, more preferably, a human of any age, including a newborn or a child. Non-human primates are subjects as well. The term subject includes domesticated animals, such as cats, dogs, etc., livestock (for example, cattle, horses, pigs, sheep, goats, etc.) and laboratory animals (for example, ferret, chinchilla, mouse, rabbit, rat, gerbil, guinea pig, etc.). Thus, veterinary uses are contemplated herein. Optionally, the subject is a subject having or suspected of having type 2 diabetes or prediabetes. Optionally, the subject is a subject exhibiting one or more symptoms of type 2 diabetes or prediabetes.

The foregoing description of some examples has been presented only for the purpose of illustration and description and is not intended to be exhaustive or to limit the disclosure to the precise forms disclosed. Numerous modifications and adaptations thereof will be apparent to those skilled in the art without departing from the spirit and scope of the disclosure.

Reference herein to an example or implementation means that a particular feature, structure, operation, or other characteristic described in connection with the example may be included in at least one implementation of the disclosure. The disclosure is not restricted to the particular examples or implementations described as such. The appearance of the phrases “in one example,” “in an example,” “in one implementation,” or “in an implementation,” or variations of the same in various places in the specification does not necessarily refer to the same example or implementation. Any particular feature, structure, operation, or other characteristic described in this specification in relation to one example or implementation may be combined with other features, structures, operations, or other characteristics described in respect of any other example or implementation.

Use herein of the word “or” is intended to cover inclusive and exclusive OR conditions. In other words, A or B or C includes any or all of the following alternative combinations as appropriate for a particular usage: A alone; B alone; C alone; A and B only; A and C only; B and C only; and A and B and C.

### Examples

### Example 1. Multi-Modal Analysis of Type 2 Diabetes in the Project Baseline Health Study

The Project Baseline Health Study (PBHS) is a prospective, multicenter, longitudinal study including participants with diverse backgrounds and representative of a wide spectrum of health. During the study, longitudinal data are collected enabling multiple modalities of deep phenotyping, including medical history, clinical laboratory tests, molecular and digital profiling (Arges et al. 2020). Previous research has analyzed the PBHS cohort to identify clinical characteristics of diabetes and prediabetes (Chatterjee et al. 2022).

Here, the clinical characterization of type 2 diabetes (T2D) in the PBHS cohort was expanded by integrating proteomics and clinical profiling to identify plasma proteins associated with diabetes. Enrichment analysis, network analysis and transcriptomics analysis was performed to determine which pathways were altered in participants with diabetes compared to normoglycemic participants. Finally, unsupervised and supervised machine learning modeling that combined proteomics and clinical data was performed to assess whether the integration of multiple modalities may improve diabetes phenotyping compared to a single data modality.

### Methods

### Participants: The Project Baseline Health Study

The PBHS is a longitudinal cohort study approved by both a central Institutional Review Board (the WCG IRB; approval tracking number 20170163, work order number 1-1506365-1) 15 and TRBs at each of the participating institutions: Stanford University, Duke University, and the California Health and Longevity Institute (Arges et al. 2020). This study included participants who met all PBHS eligibility criteria—key criteria were US residency and age ≥18 years—and consented to participate. A full description of study procedures and has been previously reported [Arges 2020].

During the study visits, questionnaires collected participants' medical history information (spanning multiple disease areas including immune, metabolic and cardiovascular, mental health, neurological, infectious and musculoskeletal) and biological samples were collected and bio-banked. Samples collected include whole blood, plasma, serum, stool, saliva, tears, urine, and facial swabs. Blood and urine samples were also submitted for standard clinical laboratory analysis, including complete blood count (CBC). Participants also underwent echocardiography and wore a Verily Study Watch (Verily Life Sciences, South San Francisco, California), which recorded acceleration data via an onboard inertial measurement unit (IMU) with a 30 Hz 3-axis accelerometer. The data included in this analysis were collected between 2017-2022.

### Analyzable Cohort

The analyzable cohort for this study consisted of 732 participants in the PBHS with available proteomics data and who maintained the same diagnosis throughout the study (unless otherwise noted).

The portion of this study involving data modeling included the subcohort of participants with complete clinical data available to enable the analysis.

Availability of proteomic data. Proteomics data were available from several participant subsets within the PBHS and were analyzed together for the present study. The subsets included the initial participants enrolled; participants with self-reported T2D or with clinical variables indicative of prediabetes or T2D risk (HbA1c, fasting blood glucose [FBG], low and high density cholesterol, triglycerides, body mass index, waist circumference), and normoglycemic participants matched based on demographics and overall physical health (specifically based on sex at birth, age, race, blood pressure, resting pulse rate, respiratory rate, average daily step count); and participants selected for PBHS substudies focused on autoimmune diseases and liver injury.

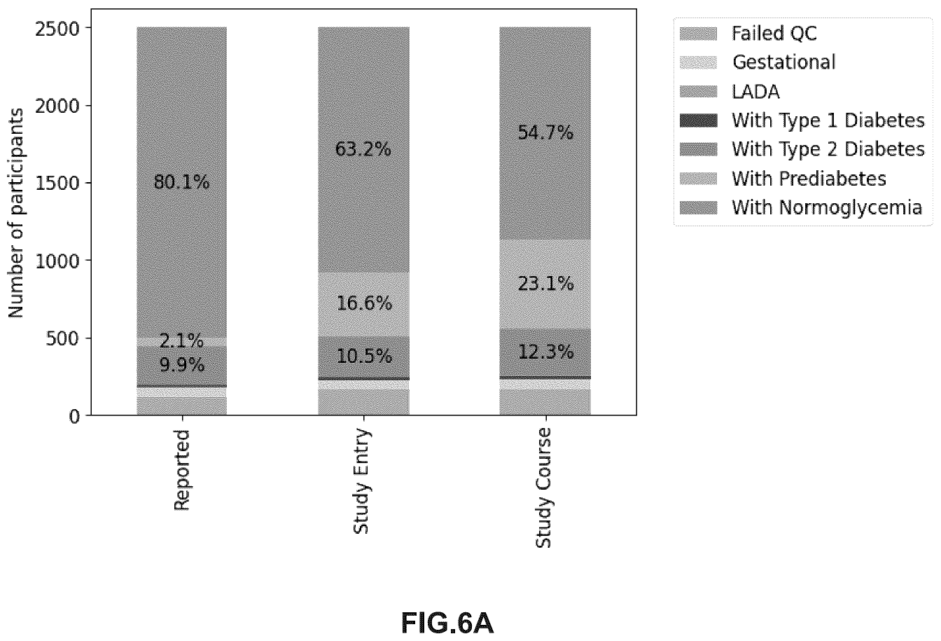

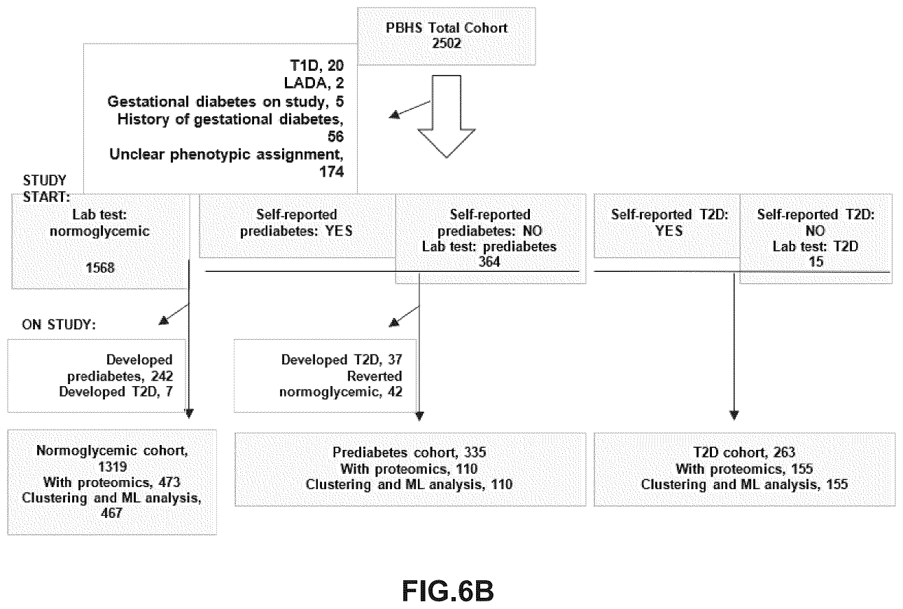

Diagnosis at study start and follow-up. Two sources of information were integrated: self-reported status and results from on-study clinical tests for HbA1c, FBG and non-fasting blood glucose (nFBG). Participants with pre-existing diagnoses of T2D or prediabetes, including those with HbA1c or blood glucose values outside of the disease's clinical range at study start, were classified according to the pre-existing diagnosis (assuming these may reflect cases of successful disease management). Participants without a diagnosis for T2D or prediabetes could be classified as ‘with T2D’ or ‘with prediabetes’ if their HbA1c or blood glucose was in the diabetic or prediabetic clinical range at study start and at the following yearly visit (diabetes defined as HbA1c≥6.5%, or FBG≥126 mg/dL or random blood glucose [RBG]≥200 mg/dL; prediabetes defined as HbA1c between 5.7%-6.4%, or FBG between 100 mg/dL-125 mg/dL) (CDC 2023). In order to monitor the maintenance of a given diagnosis or the occurrence of progression events to T2D or prediabetes, study measurements of HbA1c and blood glucose were followed. When HbA1c or blood glucose test results shifted to the diabetic or prediabetic clinical range for at least 2 study visits at any point, diagnoses were updated (Table 3, FIGS. 6A and 6B). Self-reports of initiation of diabetes medications while on the study, were marked as progression events to prediabetes or T2D depending on the indication of the medication. The following diabetes medications were considered: metformin, pioglitazone, glimepiride, exenatide, canagliflozin, empagliflozin, dapagliflozin, dulaglutide, glimepiride, glibenclamide, glipizide, glucagon, chlorpropamide, glyburide, sitagliptin, saxagliptin, linagliptin, alogliptin, semaglutide, liraglutide and insulin.

Normoglycemic participants reporting taking diabetes medications for the treatment of another condition, such as polycystic ovary syndrome (PCOS), were excluded from the analysis.

### Proteomics

### Experimental Setup and Data Acquisition

Plasma was aliquoted from whole blood samples collected in K2 EDTA tubes and plasma samples were processed through Verily's proprietary liquid chromatography-mass spectrometry (LC/MS) proteomics assay (For full details, see proteomics assay below).

### Proteomics Analysis Pipeline

According to the experimental design, each sample was processed as two technical replicates for each batch. The two technical replicates within the batch were injected in randomized non-consecutive order onto the LC-MS instrument. If the instrument performance was degrading during a batch, more than two replicates were processed. Custom code was used, unless specified otherwise.

Mass spectra were stored as proprietary ThermoFisher .raw files. The spectra were analyzed to infer peptide and protein abundances (see processing steps in inference of peptide and protein abundance section below).

### Plasma Contamination

To take into account potential biases due to different levels of plasma contamination at sample collection, contamination indices for erythrocytes, platelets and coagulation were computed (Geyer et al. 2019). Each contamination index was computed in each individual sample by summing the expression of the proteins in each contamination index protein signature (Geyer et al. 2019). Platelet and erythrocyte contamination was computed as the ratio of the sum of platelet and erythrocyte protein expression over the sum of all expressed proteins in each sample. Coagulation contamination was computed as the ratio of the sum of all expressed proteins over the sum of coagulation proteins in each sample.

The sample-specific contamination indices were added as confounding variables to the differential expression model.

### Differential Protein Expression Analysis

To identify differentially expressed (DE) proteins between individuals with T2D and normoglycemia, a linear model was built for each protein. The batch-corrected expression of each protein was modeled as a function of the diabetic phenotype, accounting for the following potential confounding factors: sex, age, race, smoking status, presence of comorbidities, statin usage, hypertension medication usage, platelet contamination, erythrocyte contamination and coagulation contamination. Participants self-reported as never smoking, formerly smoking or currently smoking, which was mapped to a discrete variable in that order. Presence of self-reported comorbidities was added as a single model term. Comorbidities were: cancer, autoimmune diseases, excluding diabetes, infectious diseases, diverticulitis, pancreatitis and pneumonia. The ols( ) function from the statsmodels.formula.api python package was used to build the linear models. The p-value associated with the coefficient of the diabetes phenotype was adjusted for multiple testing with the Benjamini-Hochberg correction (Benjamini and Hochberg 1995).

In addition, to test the stability of the differentially expressed proteins to changes in the sample composition, linear models were built for 10 random subsets of 90% of the samples, allowing resampling across the subsets. Thus, for each protein, 10 false discovery rate (FDR)-adjusted p-values, one for each of the random subsets, was obtained. Finally, a protein was considered differentially expressed if the FDR-adjusted p-value was less than 0.05 across all the random subsets.

### Gene Ontology (GO) Term Enrichment

The GO annotation from January 2023 was used to compute GO term enrichment on the DE proteins. The GO annotations were limited to terms with experimental evidence, manual and electronic annotation or inferred from sequence or structural similarity, corresponding to the following evidence codes: EXP (inferred from experiment), IDA (inferred from direct assay), IPI (inferred from physical interaction), IMP (inferred from mutant phenotype), IGI (inferred from genetic interaction), IEP (inferred from expression pattern), TAS (traceable author statement), IC (inferred by curator), IEA (inferred from electronic annotation), ISS (inferred from sequence or structural similarity). Only GO terms with at least 3 proteins represented in our data were tested for enrichment. A hypergeometric test was performed to test the enrichment for each annotated GO term within the biological process and cellular component namespaces. Up-regulated and down-regulated proteins in individuals with T2D (compared to normoglycemic) were tested for GO enrichment separately. The p-value was adjusted for multiple testing with the Benjamini-Hochberg correction (Benjamini and Hochberg 1995) separately for each namespace and each protein set. The list of all detected plasma proteins was used as the background set for the hypergeometric test.

A protein could be annotated with more than one GO term. To annotate the proteins uniquely with one GO term on a heatmap, the following custom GO slim terms were assigned in this order: lipid transport, complement activation, blood coagulation, inflammatory response, immune system process.

### Protein Analyses: Protein-Protein Interaction (PPI) Networks and Tissue Specificity

Protein-protein interactions were exported from the STRING database v11.5 (Szklarczyk et al. 2018). Only high-confidence interactions were included (minimum combined score of 500 (von Mering et al. 2005)). In addition, only PPIs between positively co-expressed DE proteins were included (Pearson's correlation coefficient between protein expression values across all participants with T2D and normoglycemia >=0.2). The resulting PPI network was finally filtered to restrict to a core of at least 2 degrees for each node. This ensured a certain level of network connectivity.

Louvain's community detection algorithm (Blondel et al. 2008) was applied on the final PPI network. Each community was annotated with the custom GO slim categories described above. The python package networkx (Hagberg, Schult, and Swart 2008) was used for network analysis.

### Expression Patterns of the Differentially Expressed (DE) Proteins

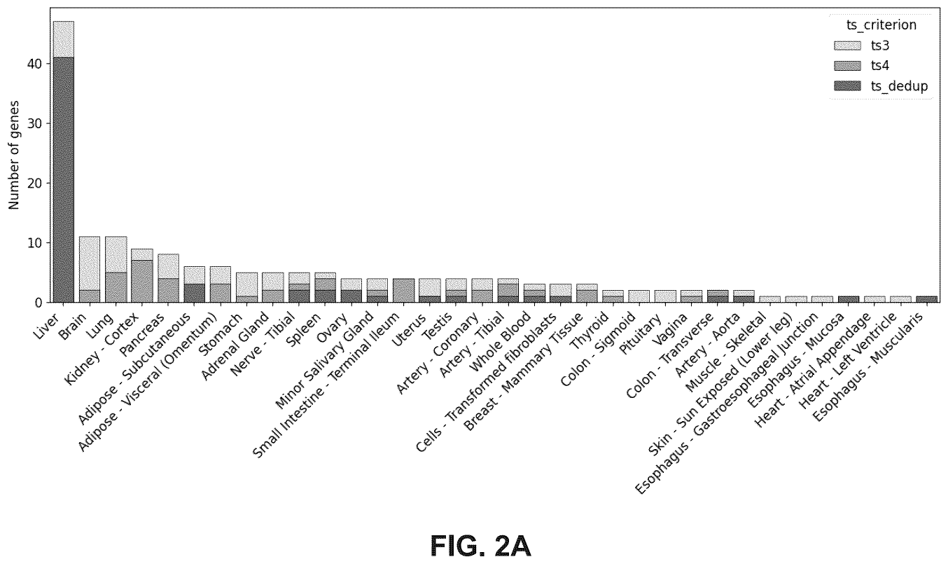

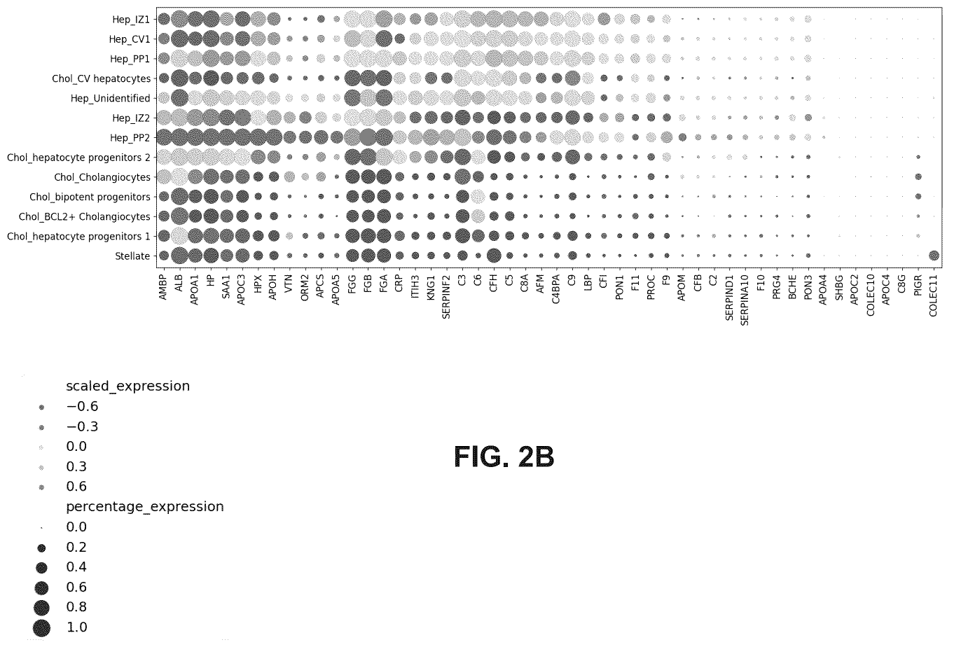

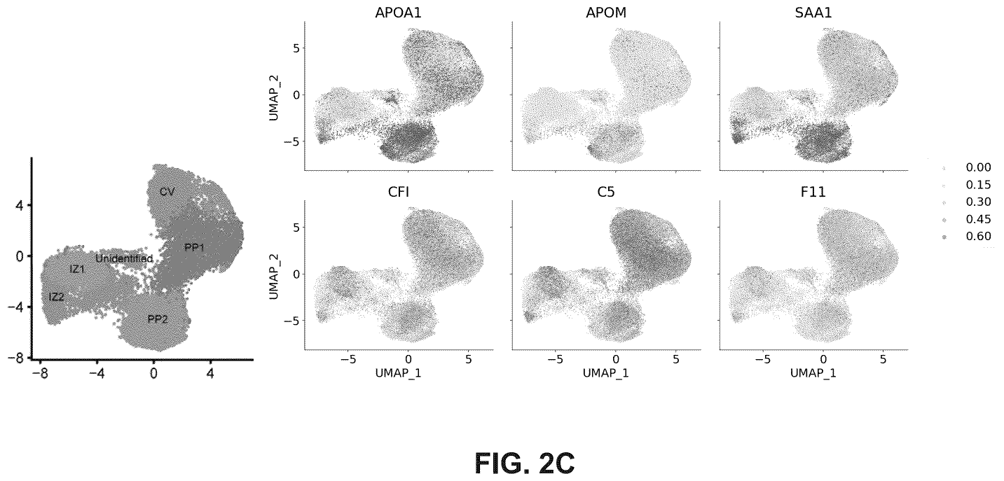

The Genotype-Tissue Expression (GTEx) database was used to examine the expression patterns of the DE proteins. Because some genes in GTEx can be specific to multiple tissues (Yang et al. 2018), tissue-specific genes encoding for DE proteins were selected using increasingly stringent tissue-specificity thresholds (tissue-specificity score>3 or >4). In addition, the tissue assignment was deduplicated by assigning the gene to the tissue with the highest tissue specificity score (FIG. 2A).

### Single Cell Liver RNA-Seq Analysis

Single-cell RNA-seq (scRNA-seq) data obtained from the liver of healthy donors was downloaded from the GSE185477 GEO study (Andrews et al. 2022) Liver cells from multiple healthy donors are pooled into the same dataset. The authors provided single cell type annotation, normalized read counts at the single cell level, and UMAP projection values. For each DE protein expressed in liver, bulk RNA-seq from GTEx (FPKM>1), the average Z-score of gene expression was computed in the liver scRNA-seq dataset scaled across the set of hepatic cell types, namely hepatocytes, cholangiocytes and stellate cells (FIG. 2B). UMAP values are taken directly from the original dataset (FIG. 2C).

### Clustering of Participants at Study Start

Clustering analysis was performed on 110 participants with prediabetes, 155 with diabetes, and 467 normoglycemic, with clinical and proteomics data at study start. Supervised principal component analysis (PCA) was performed on filtered clinical and proteomics features before clustering (see details below).

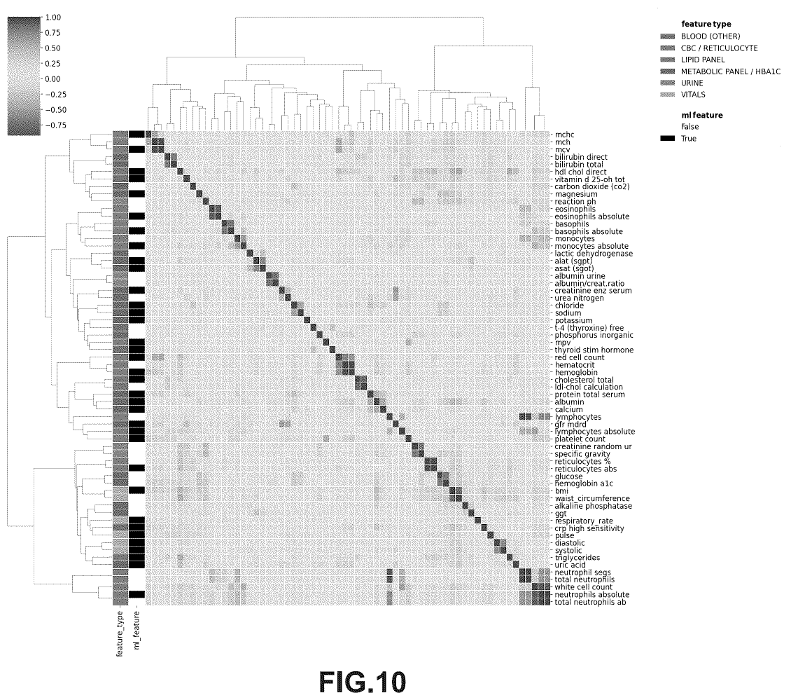

Clinical features included clinical and demographic variables (sex, age and race). Self-reported race was categorized as Asian, Black or African American, Hispanic, White or Other. Clinical features measured from standard blood and urine tests and vitals were manually curated to remove redundancy and avoid missingness. To avoid collinearity in measurements, the manual curation removed results from laboratory measurements known to be clinically related to or derived from each other and confirmed to be correlated with each other in the current cohort (Pearson correlation>0.8, FIG. 10).

The selected clinical features were concatenated to the matrix of batch-corrected expression values of DE protein, identified as described above, and used as input for PCA.

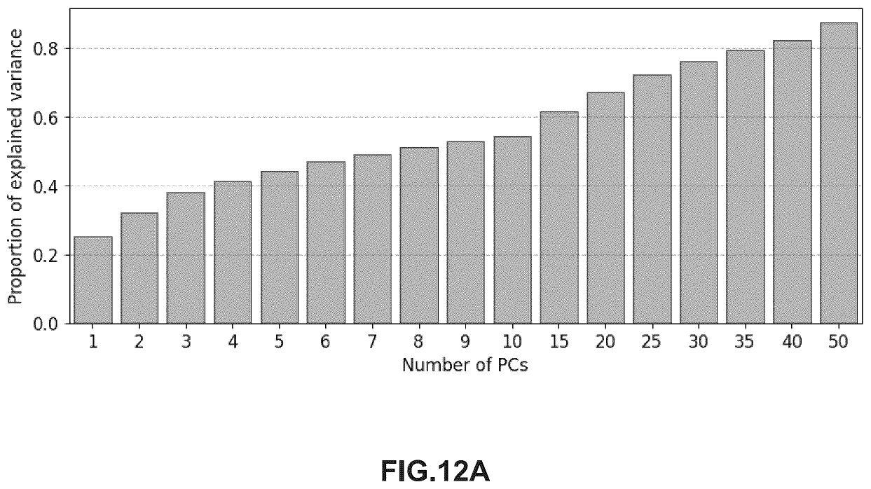

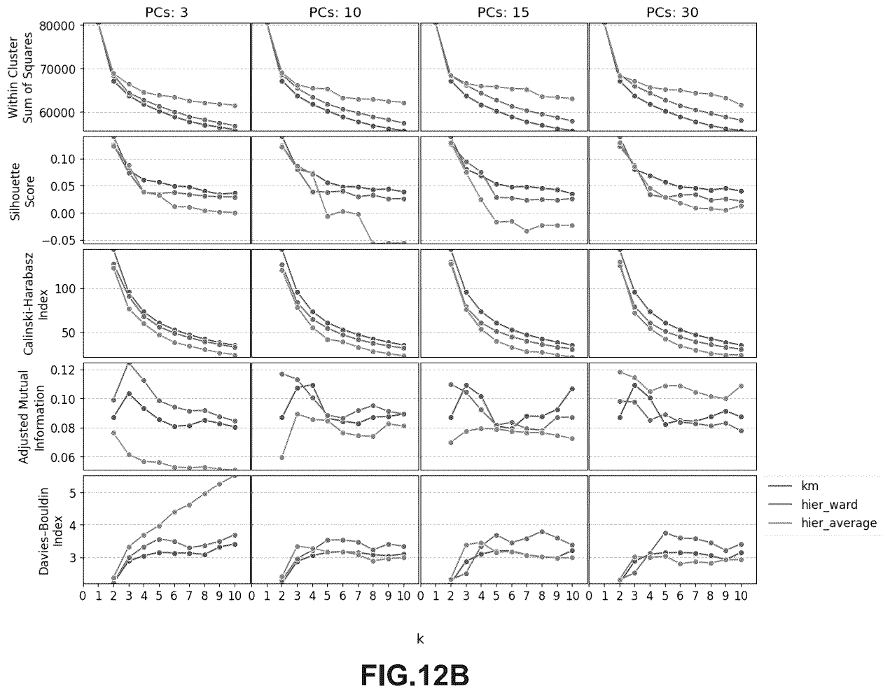

Different combinations of the number of principal components, clustering algorithms and k number of clusters were evaluated by computing commonly used clustering metrics. Specifically, the following combinations were tried:
3, 10, 15 or 30 principal components, based on the percentage of variance explained by each number of components
k-means clustering, hierarchical clustering with Ward clustering and Euclidean distance, hierarchical clustering with average clustering and Euclidean distance
k={2, 3, 4, 5, 6, 7, 8, 9, 10} clusters
   The clustering metrics computed were:
Within-cluster sum-of-squares. It measures the variability of the observations within each cluster. In general, a cluster that has a small sum of squares is more compact than a cluster that has a large sum of squares.
Silhouette score (“Silhouettes: A Graphical Aid to the Interpretation and Validation of Cluster Analysis” 1987). Mean of the Silhouette Coefficients for each sample. The score is bounded between −1 for incorrect clustering and +1 for highly dense clustering. Scores around zero indicate overlapping clusters. The score is higher when clusters are dense and well separated, which relates to a standard concept of a cluster.
Calinski-Harabasz index (Calinski and Harabasz 2007). The index is the ratio of the sum of between-clusters dispersion and of within-cluster dispersion for all clusters (where dispersion is defined as the sum of distances squared). A higher Calinski-Harabasz score relates to a model with better defined clusters.
Adjusted Mutual Information index (Vinh, Epps, and Bailey 2010). It is an adjustment of the Mutual Information (MI) score to account for chance. It accounts for the fact that the MI is generally higher for two clusterings with a larger number of clusters, regardless of whether there is actually more information shared.
Davies-Bouldin index (Davies and Bouldin 1979). This index signifies the average ‘similarity’ between clusters, where the similarity is a measure that compares the distance between clusters with the size of the clusters themselves. Zero is the lowest possible score. Values closer to zero indicate a better partition.
   Based on clustering performance across the different clustering metrics, k-means clustering was selected on 15 principal components, with k=3 (FIG. 12).
   Clustering Validation with Orthogonal Features

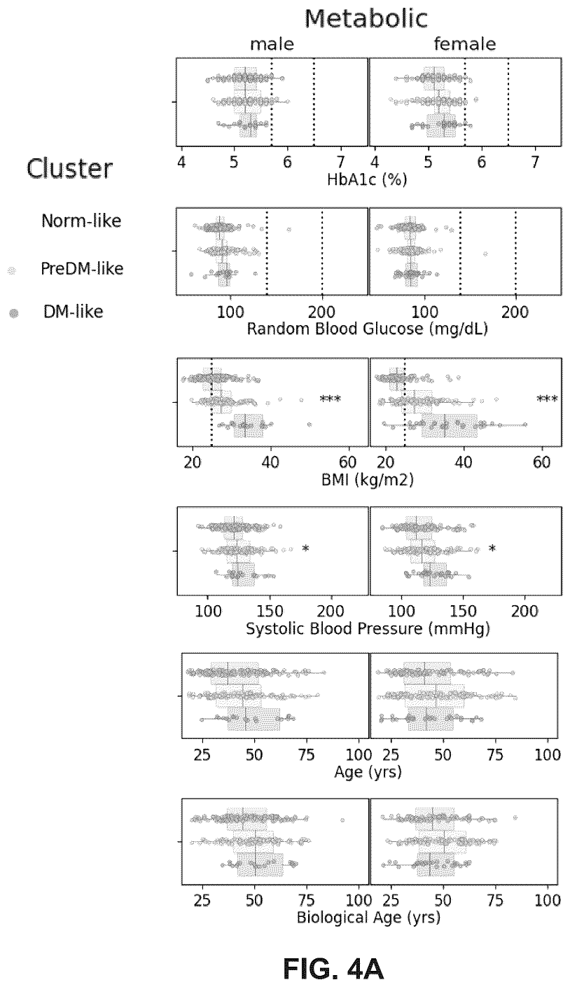

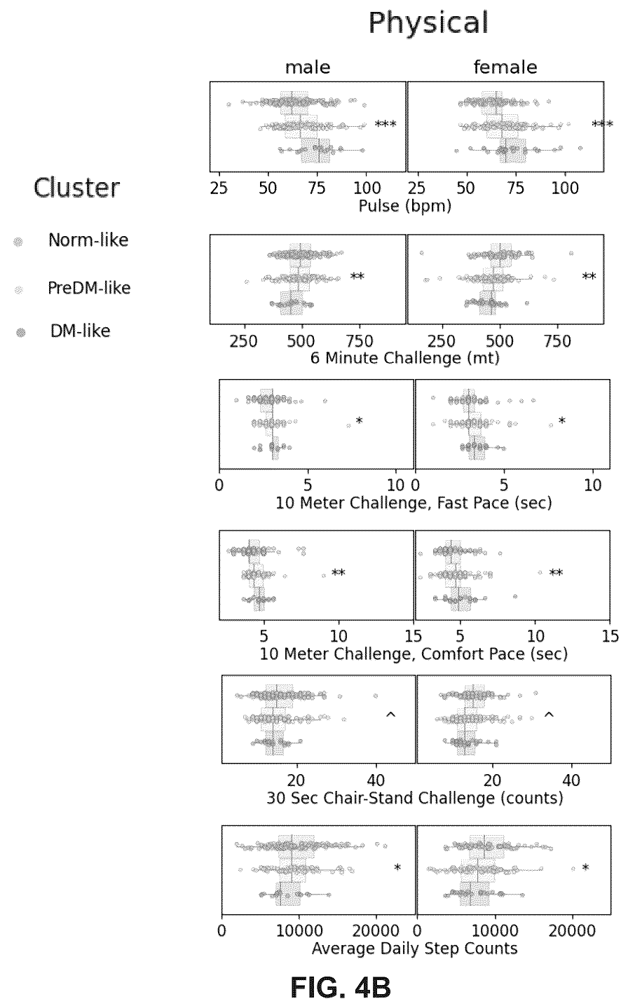

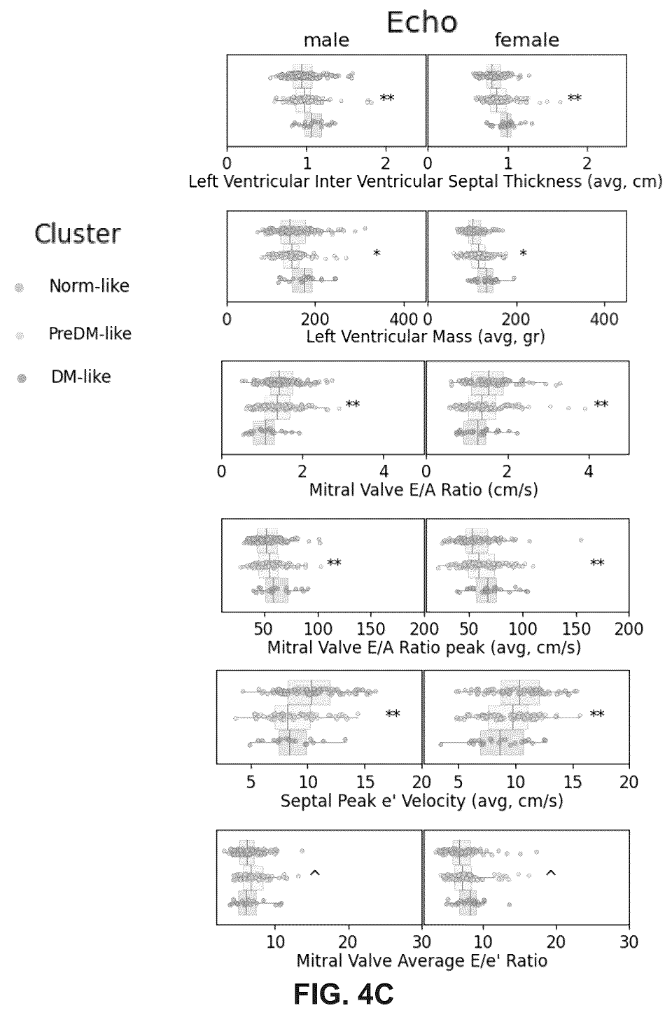

To evaluate if the clusters obtained were also related to features not included in the clustering input feature set (not related to blood work), differences of these orthogonal features was examined between diabetic-like and normoglycemic-like clusters within each clinical phenotype (FIGS. 4A-4C).

### Methylation Age

Methylated DNA was measured using the Illumina EPIC 850K array from DNA extracted from frozen, stored whole blood collected at enrollment (see (Uchehara et al. 2023) for details). DNA methylation derived ages were predicted using coefficients supplied by Horvath using a linear combination of the coefficients and the corresponding beta value in each sample (Horvath 2013). An adjustment was made for non-adult age, as described in the corresponding manuscript. Missing values were filled in using a standard value provided by the authors (see (Uchehara et al. 2023) for details).

### Physical Activity Data

As part of PBHS assessments, participants underwent standard physical performance challenges, including: six-minute walk test, ten-meter walk tests (fast pace and comfortable pace), 30-second chair stand. In addition, the average number of daily steps during daily living was computed using the data collected from the Verily Study Watch [Popham 2023 ref]. For each day of the week, Monday to Sunday, the median number of daily steps on that day of the week was computed over 90 days. Only days with at least 720 minutes of watch wearing time were included in the median calculation. The medians were averaged to obtain an average daily step count.

### Echocardiographic Measurements

Each study site performed echocardiography with quality control by the Duke Clinical Research Institute Imaging Core Laboratory. Images were analyzed according to best practices and the American Society of Echocardiography recommendations for chamber quantification and assessment of diastolic dysfunction (detailed methods previously published, Cauwenberghs 2023).

### Machine Learning Models of Type 2 Diabetes

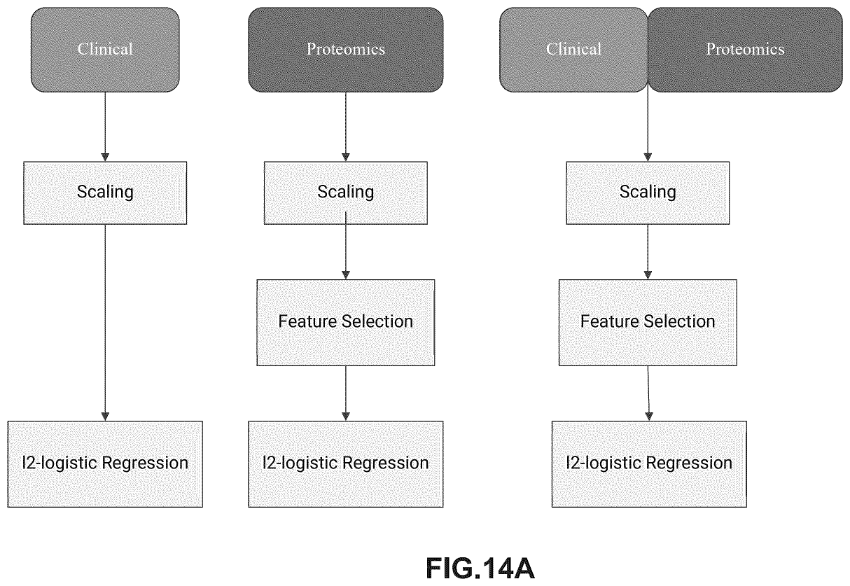

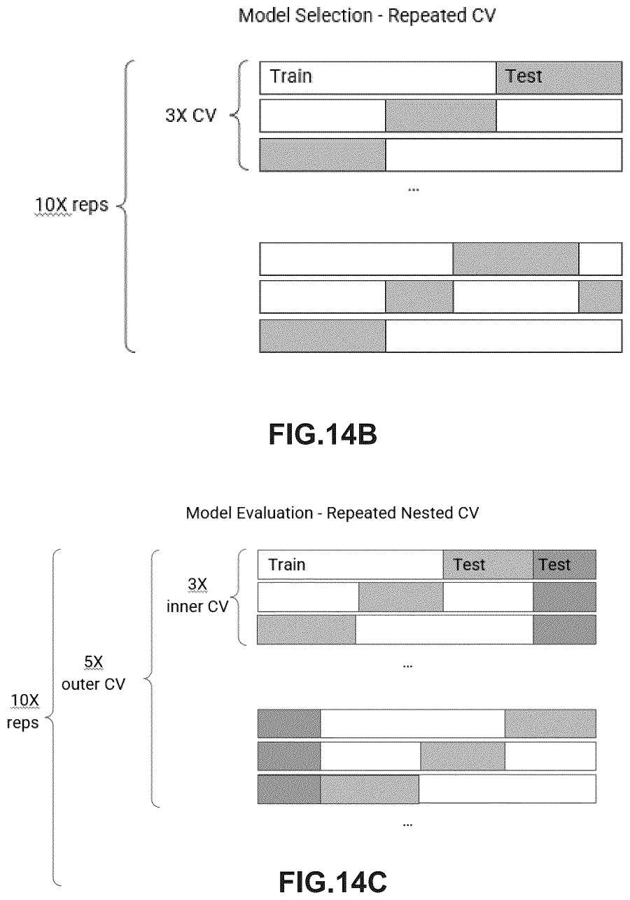

Three T2D classification models were built using three different sets of input features (FIG. 14A) for 155 participants with T2D and 467 participants with normoglycemia who had both clinical and proteomics data: clinical only, proteomics only and clinical and proteomics combined. Input features are as follows:
Clinical only: clinical features were selected based on the correlation-based curation described for the clustering analysis (see above). HbA1c and blood glucose were omitted. This feature set included 20 features.
Proteomics only: protein expression of all detected plasma proteins, except for albumin, scaled by computing the min-max normalization of non-batch-corrected expression within each sample. Non-batch-corrected protein expression was used to minimize data leakage. Albumin was omitted because its expression was above 2.5 standard deviations from the sample mean expression in most samples. This feature set included 289 features.
Clinical and proteomics combined: which included both clinical and proteomics datasets concatenated. This feature set included 310 features.

Different preprocessing steps were performed depending on the input dataset. While feature scaling with standard scaling was applied to all datasets, feature selection was applied only to the proteomics-only dataset and to the combined dataset to reduce the dimensionality of the data. For feature selection, the top k features with the highest ANOVA F-score were selected. The k number of features was tuned as a hyperparameter (k={100, 150, 200}). Finally, a ridge logistic regression classifier was trained on the selected features. Ridge logistic regression was used because of its intuitiveness, interpretability, and ability to include multiple collinear features (Cessie and Van Houwelingen 1992). The regularization strength was tuned as a hyperparameter (C={0.01, 0.03, 0.07, 0.18, 0.46, 1.21, 3.16}, logspace search between −2 and 0.5 with step 7). All the steps were embedded into a pipelineo object using the python scikit-learn framework (Pedregosa et al., n.d.).

Two different cross-validation (CV) designs were used for model selection and model evaluation (Krstajic et al. 2014)(FIGS. 14B and 14C):
Model selection for model interpretation with repeated CV (FIG. 14B). Ten (10) repeats of 3-fold CV were performed on the entire dataset to find the best combination of hyperparameters for each set of input feature sets, and used cross-entropy as the criterion to select the final model. To obtain the final logistic regression weights, the whole model was retrained on the entire dataset, using the selected hyperparameters. The best hyperparameters for each model were:
   Clinical only: C=0.32
   Proteomics only: C=0.07, k=150
   Clinical and proteomics combined: C=0.018, k=150

Model evaluation with repeated nested CV (FIG. 14C). To evaluate the final model, two loops of CV were performed, one outer 5-fold CV loop, where 20% of the data is used as test set, and an inner 3-fold CV loop, where one third of the remaining 80% of the data was used as test set for hyperparameter tuning, and two thirds were used for training. For each of the 5 folds, a different optimal set of hyperparameters might be chosen; this is still acceptable to evaluate the model selected with repeated CV, as long as the parameter search space is the same (Krstajic et al. 2014). The nested CV iterations are repeated 10 times, each time with a different random seed, to obtain a distribution of performance metrics.

Model Interpretation with SHAP Values

Feature importance for the diabetes prediction model using the combined dataset was assessed by analyzing the SHapley Additive exPlanations (SHAP) values (Lundberg and Lee 2017) in the prediabetic population at study start. The SHAP values for the prediabetic participants were computed from the model trained on the entire cohort of normoglycemic and diabetic participants, as defined above. Examining the SHAP values associated with a model can reveal what features are driving the model prediction for each observation in the dataset.

To summarize the SHAP values of the protein features, SHAP values for groups of functionally related proteins were added together. This was possible because of the additive nature of SHAP values (Lundberg and Lee 2017). The groups of functionally related proteins were manually curated from GO term annotation and domain expert knowledge (Table 1).

TABLE 1
Groups of functionally related proteins
used to aggregate SHAP values.

| Protein Function | Protein |
|---|---|
| prot_blood_coagulation | PROS1 |
| prot_blood_coagulation | FGB |
| prot_blood_coagulation | F9 |
| prot_complement | C7 |
| prot_complement | C5CFI |
| prot_complement | VTN |
| prot_complement | CFB |
| prot_complement | C4BPA |
| prot_hdl | CETP |
| prot_hdl | APOD |
| prot_hdl | APOC1 |
| prot_hdl | APOM |
| prot_hdl | APOA1 |
| prot_ldl | APOH |
| prot_ldl | APOC4 |
| prot_ldl | APOC3 |
| prot_ldl | APOC2 |
| prot_ldl | APOA4 |
| prot_ldl | LCAT |

### Proteomics Assay

Plasma proteins were prepared through a proteomics pipeline, utilizing robotic liquid handling and validated plasma preparation kits to achieve high throughput processing, consistency, and scale. For each plasma sample, 2 microliters were denatured with trypsin/Lys-C protease and the subsequent peptides were desalted and dried down in a vacuum concentrator. Dried pellets were dissolved in 40 microliters of 0.1% (v/v) formic acid, then peptide concentrations were normalized to 1 microgram per microliter and combined with iRT standard peptides (1:20 v/v). 5 micrograms of each sample was randomly injected in duplicate onto a customized microflow high-resolution liquid chromatography-mass spectrometry (LC-MS) setup. Mass spectra were acquired in data-independent acquisition (DIA) mode for accurate and reproducible quantification. Raw data files were saved locally and on the cloud for downstream analysis.

### Inference of Peptide and Protein Abundance

Peptide abundance was inferred through several processing steps:
1. The .raw files from the MS analysis were converted to .mzML format using msconvert from ProteoWizard (Adusumilli and Mallick 2017). In addition, .mzML files were centroid normalized for compatibility with following steps.
2. Dia-NN v1.8.1 (Demichev et al. 2020) is executed in library-free mode to generate preliminary peptide abundances. The plasma proteome from the Human PeptideAtlas (Deutsch et al. 2021) build 2022-05 was used to restrict the search space to plasma protein sequences.
   Dia-NN was executed with the following options:
   f file.mzML-fasta PeptideAtlas.fasta-fasta-search-gen-spec-lib-gen-fr-restriction-mass-acc-msl 5-mass-acc 15-min-pr-mz 400-max-pr-mz 880-window 6-restrict-fr-no-fr-selection-no-norm-no-maxlfq-individual-reports
3. A subset of samples was analyzed jointly to generate the spectral library with Dia-NN. Specifically, the first available replicate of each sample was selected from each participant's entry visit. Dia-NN parameters were the same as in the previous step.
4. Each replicate sample was reprocessed independently using the newly generated spectral library. Dia-NN parameters were the same as in the previous steps, except now the parameters related to the library-free search, namely-fasta PeptideAtlas.fasta-fasta-search-gen-spec-lib-gen-fr-restriction, are replaced with -lib library.tsv -no-prot-inf
   Peptide abundances quantified with Dia-NN were filtered, normalized and aggregated to protein abundances through several processing steps:
1. Quality metrics were computed and used to filter samples and precursors used for following analyses. Specifically:
   a. When a batch was repeated only the latest batch was used for analysis
   b. Only replicates with more than 2500 precursors were used for analysis
   c. Only samples with at least two valid technical replicates were used for analysis. In the case of more than two technical replicates per sample, the first two were used for analysis
   d. Proteins needed to have Dia-NN library q-value<0.01
   e. Proteins needed to have Dia-NN q-value<0.05 in both replicates in at least 100 samples
   f. Precursors needed to be reproducible between replicates, coefficient of variation<0.2 for all samples
   g. Microbial proteins, contaminants and Ig variable chain proteins were not included in downstream analyses
2. Mass spectrometer performance might degrade as samples are loaded during a batch run. This might cause precursor expression drift as a function of when the sample was loaded on the instrument. A third-order polynomial regression for the log-transformed precursor expression was fit on the run order for each precursor in each batch, and predictions were regressed out to the median to adjust for temporal bias.
3. Non-log transformed normalized precursor quantities were summed up to compute protein abundances within each replicate.
4. Protein quantities were log-transformed again and averaged between technical replicates to obtain protein quantities at the sample level. Missing values in one replicate were imputed with values from the other replicate.
5. Finally, averaged protein quantities were corrected for batch effects using a python implementation of the combat method (Behdenna et al. 2020; Johnson, Li, and Rabinovic 2007).

### Use of Clinical Data to Clustering Participants at Study Start

The following clinical data (from standard laboratory tests) and vitals were curated to remove redundancy avoiding missingness:
Only laboratory results from blood were included, not urine
Only absolute blood cell counts were included in the model, blood cell percentages were excluded
Between BMI and waist circumference, only BMI was included
Only total cholesterol and HDL cholesterol were included, while calculated LDL cholesterol was excluded
Between hematocrit and hemoglobin, only hemoglobin was included
   Of these, only clinical features with significant association with diabetes were included in the clustering. Association with diabetes was tested by building linear models where each clinical variable was modeled as a function of the diabetes phenotype, accounting for potential confounding factors, including sex, age, race, smoking status, comorbidities, statin usage and hypertension medication usage. The p-value associated with the coefficient of the diabetes phenotype was adjusted for multiple testing with the Benjamini-Hochberg correction (Benjamini and Hochberg 1995)(FIG. 10).
   Clustering Validation with Orthogonal Features
   These are the definitions of the physical performance tests included in PBHS assessments:
Six-Minute Walk Test: the subject is instructed to walk for 6 minutes at a comfortable pace. The distance walked is measured.
Ten-Meter Walk Test (fast pace): the subject is instructed to walk 10 meters at a fast pace. The time needed to complete the challenge is measured.
Ten-Meter Walk Test (comfortable pace): the subject is instructed to walk 10 meters at a fast pace. The time needed to complete the challenge is measured.
30-Second Chair Stand: the subject is instructed to sit on a chair without armrests and stand repeatedly for 30 seconds. The number of times the subject switches between sitting and standing is measured.

### Results

### Study Population and Molecular Data Generation

Of 2502 participants in the originating total PBHS cohort, 174 were initially excluded due to inconclusive reports for phenotypic assignment, and 83 due to having conditions incompatible with this study (LADA, 2; T1D, 20; history of gestational diabetes, 56; gestational diabetes on study, 5) (Table 2 and Table 3 below, FIG. 6A, 6B)

| | | | | Evaluable, N = 1915 | | | | | | Evaluable with Proteomics at Start, n = 698 | | | | | |
|---|---|---|---|---|---|---|---|---|---|---|---|---|---|---|---|
| | | Total PBHS | | Normoglycemic | | Prediabetes | | T2D | | Normoglycemic | | Prediabetes | | T2D | |
| Characteristic | | N = 2502 | | n = 1319 | | n = 335 | | n = 263 | | n = 473 | | n = 110 | | n = 115 | |
| Sex, n | Male | 1375 | (55.0) | 712 | (54.0) | 182 | (54.3) | 132 | (50.2) | 220 | (46.5) | 57 | (51.8) | 81 | (52.3) |
| (%) | Female | 1127 | (45.0) | 607 | (46.0) | 153 | (45.7) | 131 | (49.8) | 253 | (53.5) | 53 | (48.2) | 74 | (47.7) |
| Age, n | 18-29 | 398 | (15.9) | 319 | (24.2) | 10 | (3.0) | 5 | (1.9) | 112 | (23.7) | 1 | (0.9) | 4 | (2.6) |
| (%) | 30-39 | 451 | (18.0) | 317 | (24.0) | 28 | (8.4) | 18 | (6.8) | 110 | (23.3) | 11 | (10.0) | 10 | (6.5) |
| | 40-49 | 411 | (16.4) | 232 | (17.6) | 42 | (12.5) | 40 | (15.2) | 93 | (19.7) | 17 | (15.5) | 28 | (18.1) |
| | 50-59 | 442 | (17.7) | 186 | (14.1) | 75 | (22.4) | 60 | (22.8) | 76 | (16.1) | 25 | (22.7) | 36 | (23.2) |
| | 60-69 | 399 | (15.9) | 145 | (11.0) | 77 | (23.0) | 76 | (28.9) | 55 | (11.6) | 28 | (25.5) | 49 | (31.6) |
| | 70+ | 401 | (16.0) | 120 | (9.1) | 103 | (30.7) | 64 | (24.3) | 27 | (5.7) | 28 | (25.5) | 28 | (18.1) |
| Race, n | White | 1590 | (63.5) | 883 | (66.9) | 200 | (59.7) | 148 | (56.3) | 310 | (65.5) | 64 | (58.2) | 87 | (56.1) |
| (%) | Black/African | 400 | (16.0) | 138 | (10.5) | 78 | (23.3) | 77 | (29.3) | 59 | (12.5) | 28 | (25.5) | 47 | (30.3) |
| | American | | | | | | | | | | | | | | |
| | Asian | 272 | (10.9) | 147 | (11.1) | 38 | (11.3) | 22 | (8.4) | 58 | (12.3) | 12 | (10.9) | 12 | (7.7) |
| | Hispanic | 88 | (3.5) | 54 | (4.1) | 9 | (2.7) | 7 | (2.7) | 12 | (2.5) | 4 | (3.6) | 3 | (1.9) |
| | Mixed | 70 | (2.8) | 45 | (3.4) | 4 | (1.2) | 3 | (1.1) | 16 | (3.4) | 1 | (0.9) | 2 | (1.3) |

| | | | | | | | | | | | | | |
|---|---|---|---|---|---|---|---|---|---|---|---|---|---|
| American | 31 | (1.2) | 21 | (1.6) | 2 | (0.6) | 1 | (0.4) | 6 | (1.3) | 1 | (0.9) | 0 |
| Indian/Alaskan | | | | | | | | | | | | | |

| | | | | | | | | | | | | | |
|---|---|---|---|---|---|---|---|---|---|---|---|---|---|
| Hawaiian/ | 28 | (1.1) | 14 | (1.1) | 3 | (0.9) | 3 | (1.1) | 9 | (1.9) | 0 | 2 | (1.3) |
| Pacific Islander | | | | | | | | | | | | | |
| Other | 23 | (0.9) | 17 | (1.3) | 1 | (0.3) | 2 | (0.8) | 3 | (0.6) | 0 | 2 | (1.3) |

| | | | | | | | | | | | | | | |
|---|---|---|---|---|---|---|---|---|---|---|---|---|---|---|
| RBG, mean (SD) | 97.7 | (36.3) | 87.4 | (11.2) | 93.8 | (13.8) | 150.3 | (70.0) | 88.0 | (12.8) | 95.7 | (15.0) | 153.0 | (68.8) |
| HbA1c, mean (SD) | 5.7 | (1.0) | 5.2 | (0.3) | 5.9 | (0.2) | 7.5 | (1.8) | 5.2 | (0.3) | 5.9 | (0.3) | 7.4 | (1.7) |
| BMI, mean (SD) | 28.4 | (6,8) | 26.8 | (5.8) | 29.2 | (6.5) | 43.3 | (7.9) | 27.2 | (6.0) | 30.7 | (6.5) | 34.6 | (7.4) |
| Comorbidities, n (%) | 988 | (39.5) | 498 | (37.8) | 145 | (43.3) | 104 | (39.5) | 171 | (36.2) | 39 | (35.5) | 39 | (25.2) |
| Hypertension, n (%) | 682 | (27.3) | 194 | (14.7) | 122 | (36.4) | 164 | (62.4) | 76 | (16.1) | 49 | (44.5) | 101 | (65.2) |
| Hypertension | 596 | (23.8) | 146 | (11.1) | 106 | (31.6) | 164 | (62.4) | 56 | (11.8) | 40 | (36.4) | 99 | (63.9) |
| medication, n (%) | | | | | | | | | | | | | | |
| Statins, n (%) | 418 | (16.7) | 90 | (6.8) | 92 | (27.5) | 124 | (47.1) | 35 | (7.4) | 38 | (34.5) | 69 | (44.5) |

Table 2. Demographic breakdown of study cohort. Summary statistics were computed for the entire PBHS cohort and for the PBHS participants with proteomics data generated from plasma collected during the initial study start visit. Clinical and medication status was evaluated at study start. Comorbidities were: cancer, autoimmune diseases, excluding diabetes, infectious diseases, diverticulitis, pancreatitis and pneumonia.

TABLE 3
Reclassification of diabetes based on HbA1C and FBS.

| | | | With | With | With Type 1 | With Type 2 |
|---|---|---|---|---|---|---|
| Diagnosis | Gestational | LADA | Normoglycemia | Prediabetes | Diabetes | Diabetes |
| Reported | 56 | 2 | 2004 | 53 | 20 | 248 |
| Study Entry | 56 | 2 | 1580 | 414 | 20 | 263 |
| Study Course | 61 | 2 | 1368 | 577 | 20 | 307 |

By complementing self-reported diagnoses with on-study laboratory results, and after excluding those whose diagnoses shifted on study, the evaluable population consisted of 1319 participants in the normoglycemic cohort, 335 with prediabetes and 263 with T2D (Table 2, FIG. 6A). Of them, plasma samples for proteomic analyses were available for 473, 110 and 115 participants, respectively.

The population with T2D generally had a higher proportion of male sex, Black race, hypertension, and hypertension medications and were older with higher RBG and HbA1c than the overall population. The group with prediabetes also generally had a higher proportion of Black participants, participants with hypertension, hypertension medications and older than the overall population (Table 2). LC/MS (Liquid Chromatography/Mass Spectrometry) proteomics was performed on plasma samples collected at study start from 698 participants (FIG. 6B). The trends in differences between our study populations and the overall populations were consistent between the entire set of participants and those for whom LC/MS proteomics data were generated at study start.

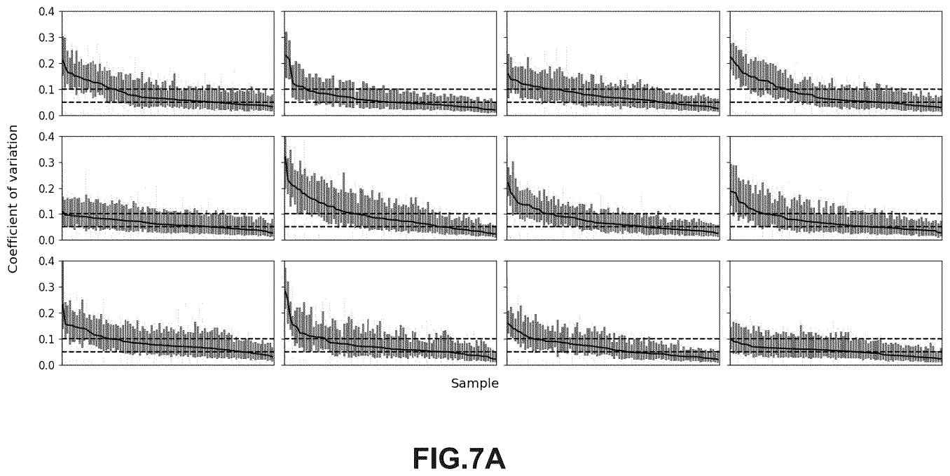

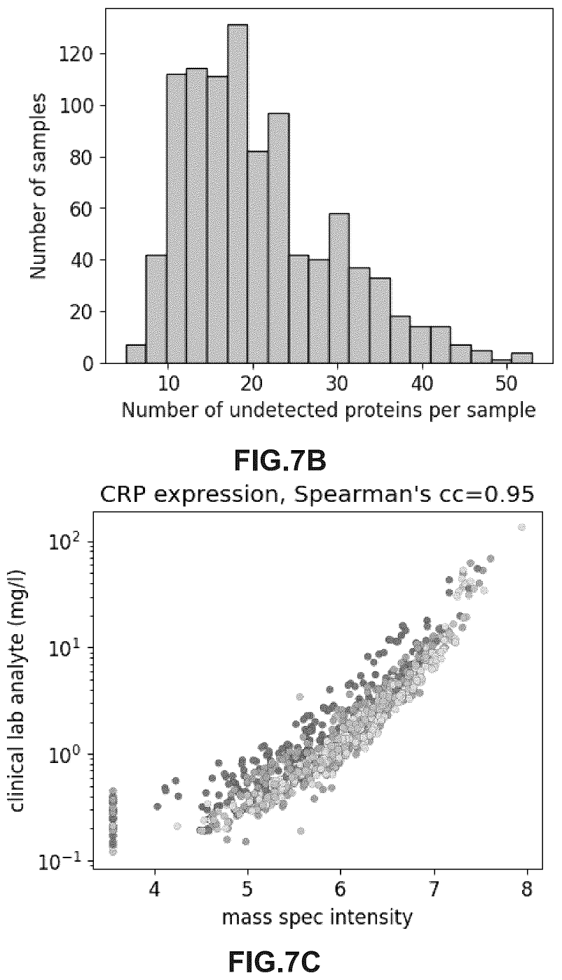

The quality of LC/MS data was assessed via commonly computed quality metrics. In particular, a median coefficient of variation of 0.07 and an average of 20 missing proteins across all samples was observed (FIG. 7A, 7B). Moreover, high correlation between C-Reactive Protein quantified by LC/MS and by standard clinical blood test was observed (Spearman's correlation coefficient=0.96, FIG. 7C).

Participants with T2D had Upregulation in Inflammation-Related Proteins

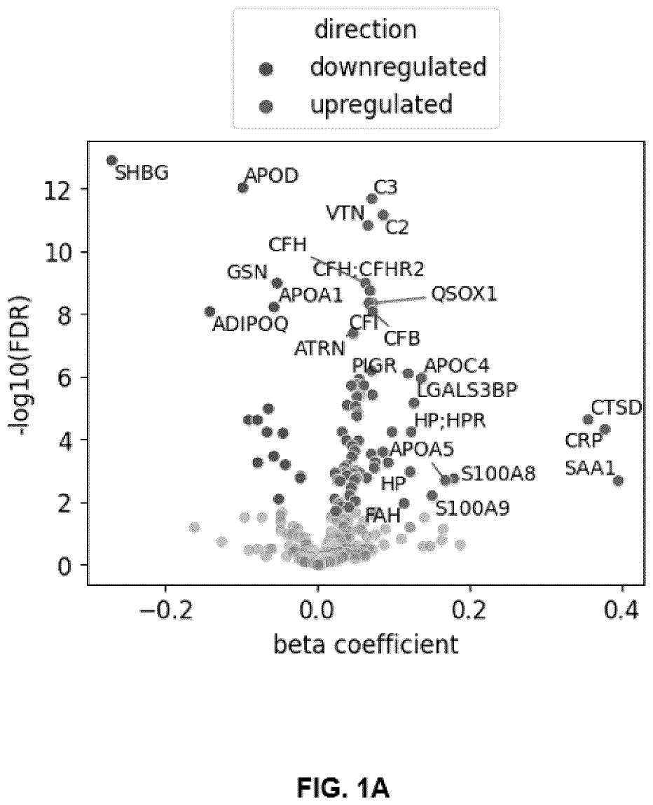

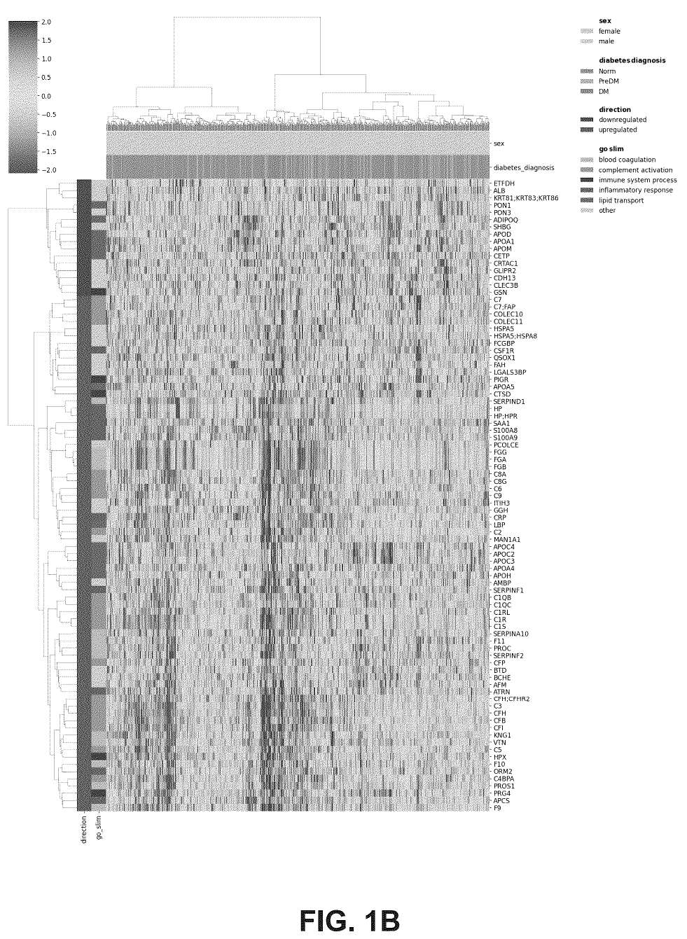

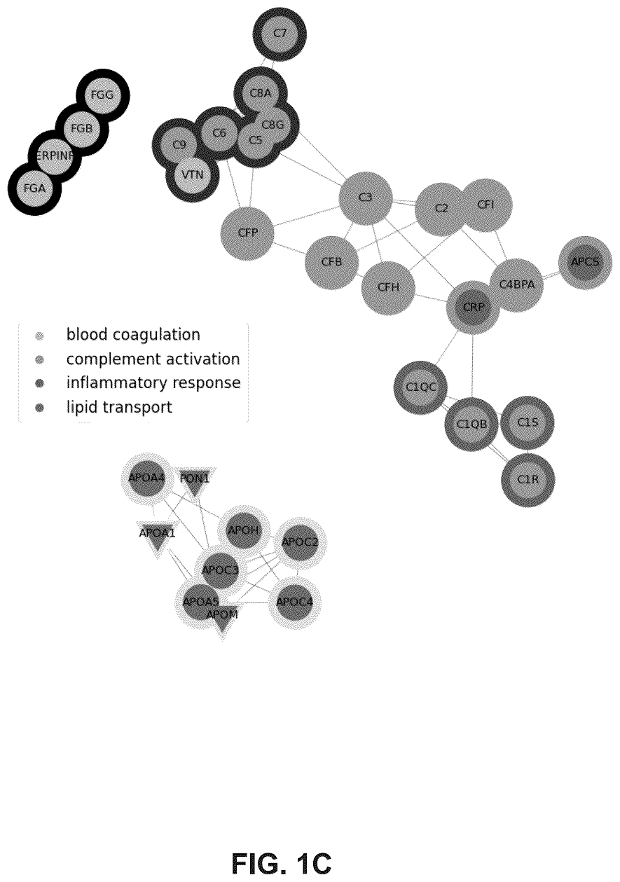

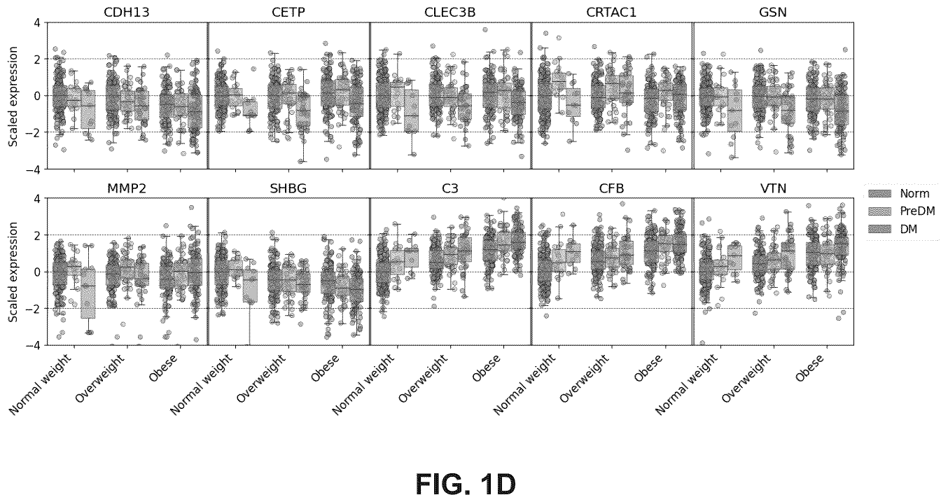

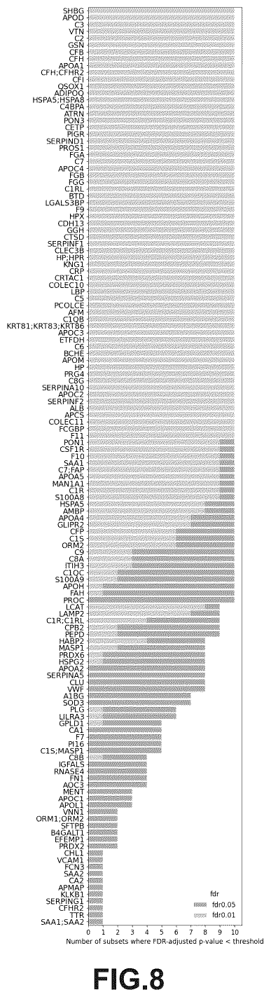

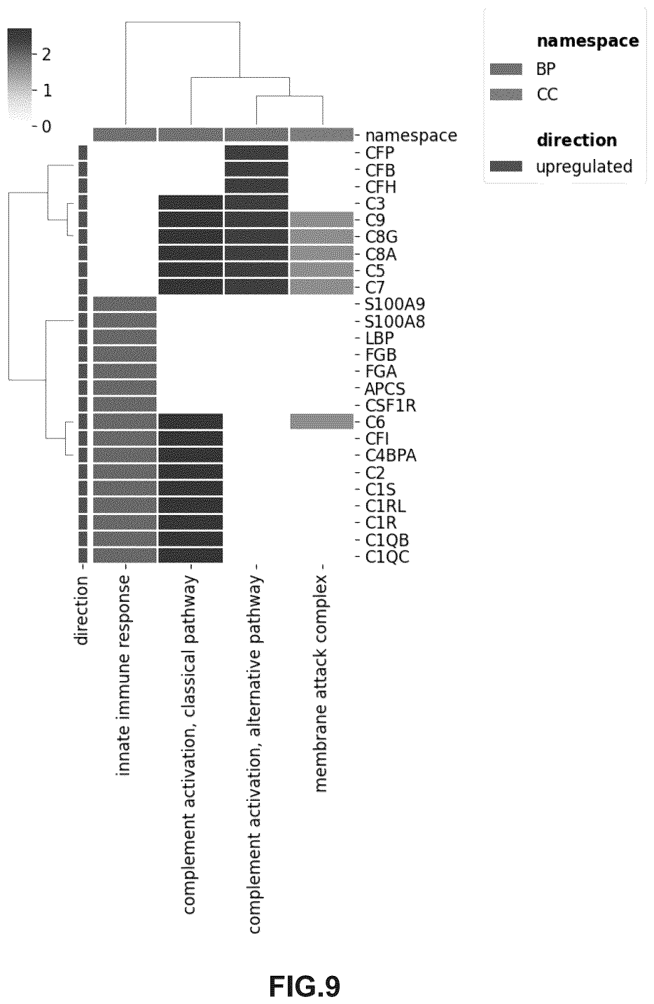

To characterize the circulating proteome in participants with diabetes, protein expression was compared between plasma samples of participants with T2D and normoglycemia. After QC filtering (Methods), a total of 289 proteins were detected across all samples. Of these, 87 differentially expressed (DE) proteins were identified (FIG. 1A, 1), after adjusting for demographic and clinical confounding variables, proteomics batch and plasma contamination and correcting for multiple testing and stability (FDR<=0.05 across all 10 bootstrapped samples, FIG. 8, see Methods). Most DE proteins, 71 (82%) showed higher expression levels in participants with diabetes, while only 16 (18%) had lower expression. Gene Ontology (GO) enrichment analysis showed that proteins involved in the complement system were more abundant in participants with T2D, while no significant enrichment was found for less abundant proteins, although many of these low-expression proteins were involved in lipid transport, especially high-density lipoproteins (HDLs) (FIG. 1B, FIG. 9).

Protein-Protein Interactions (PPIs) were Analyzed from the STRING Database
(McEnerney et al. 2017; Szklarczyk et al. 2018) for this set of DE proteins. There were four main DE protein complexes found in the PPI network of DE proteins: two complement sub-complexes, a blood coagulation complex and an apolipoprotein complex, consistent with the GO enrichment results (FIG. 1C). Interestingly, both upregulated (LDL) and downregulated (HDL) apolipoproteins are part of the same PPI community, since some of them, such as apolipoprotein C, exchange freely between lipoprotein complexes (Feingold 2021).

### DE Proteins are Secreted by the Liver and Exhibit Zonation Expression Patterns

Most DE proteins were liver-synthesized and secreted (“The Synthesis and Secretion of Plasma Proteins in the Liver” 1978) (FIG. 2A). In order to further characterize a potential relationship of DE plasma proteins with liver dysfunction in T2D, their spatial expression patterns were investigated using a single cell RNA-seq liver atlas from healthy donors (Andrews et al. 2022).

Most of the DE proteins encoded for by genes expressed in liver are preferentially transcribed in hepatocytes, with few notable exceptions, including polymeric immunoglobulin receptor (PIGR) expressed in cholangiocytes, and mannan binding lectin (MBL)-associated serine protease type 1 (MASP1) and collectin subfamily member 11 (COLEC11) expressed in stellate cells (FIG. 2B).

The genes expressed in hepatocytes reveal zonation specific transcriptional patterns. In particular, apolipoprotein genes and blood coagulation genes are more expressed in periportal and interzonal hepatocytes (PP2 and IZ2), while complement genes are more expressed in interzonal and central vein hepatocytes (IZ1, CV1, PP1) (FIG. 2B).

### Clustering Reveals Phenotypic Profiles Beyond Diagnosis

Clinical and proteomics data was combined to explore whether participants could be identified with normoglycemia and prediabetes presenting diabetic features beyond HbA1c and blood glucose.

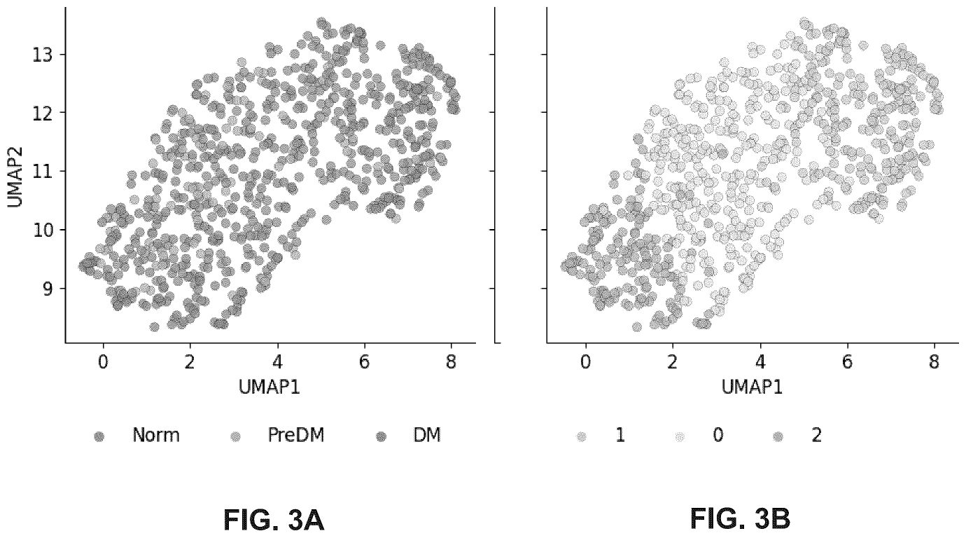

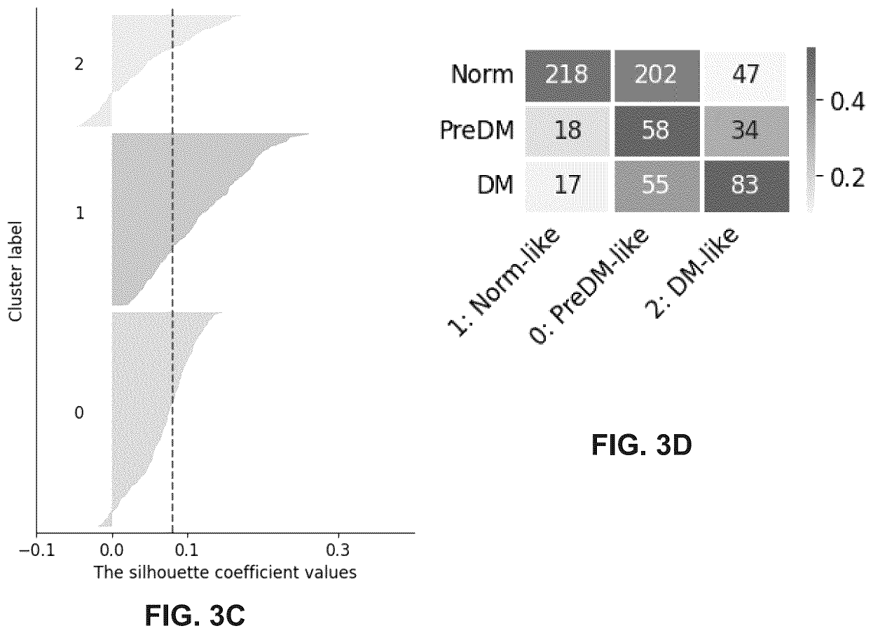

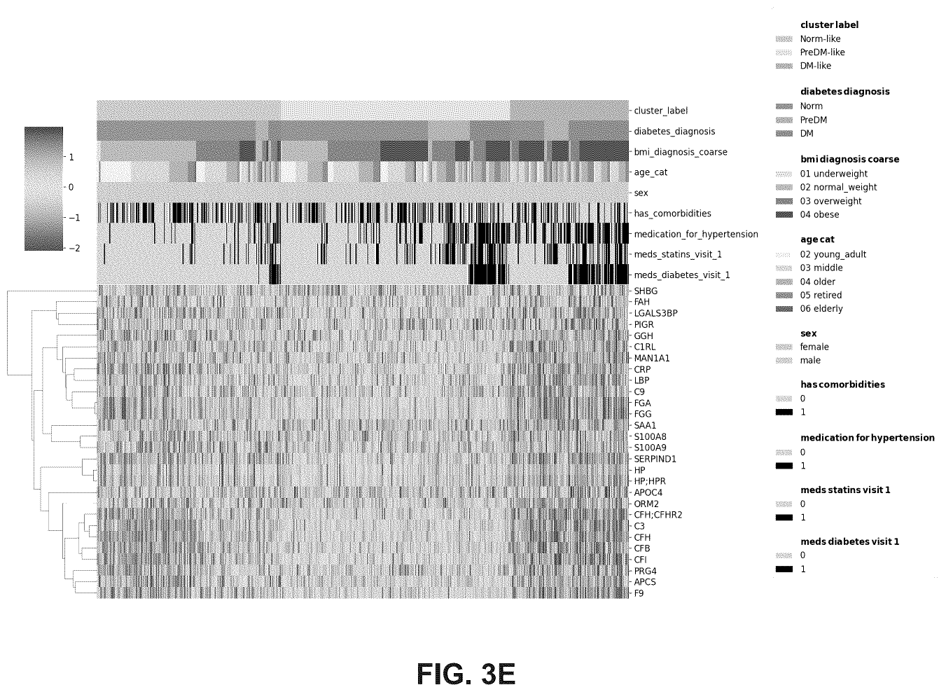

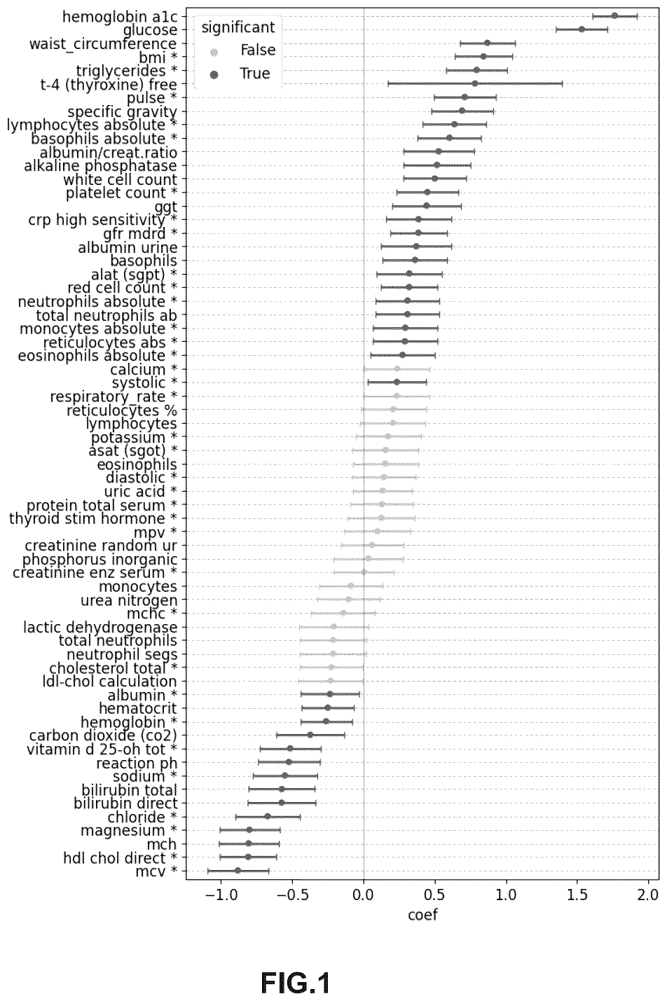

In particular, clinical features measured from standard blood tests and vitals, removing highly correlated features, were focused on (FIG. 10, Methods). In addition, clinical features were filtered for their association with diabetes in the analysis cohort (FIG. 11, Methods), and proteomics features were filtered for the DE proteins identified as described above. Supervised PCA based on these selected features showed participants follow a gradient in the projected UMAP (Uniform Manifold Approximation and Projection) space, rather than clearly defined phenotypic clusters (FIG. 3A). It was observed that the first two principal components were already explaining 30% of the variance, and the first fifteen components were capturing almost 60% of the variance (FIG. 12A). Then, several clustering algorithms were applied, different number of principal components for dimensionality reduction before clustering, and different number of clusters k to explore how unsupervised clusters relate to the clinically defined phenotypes (FIGS. 12A-12C, Methods). K-means clustering with 3 clusters (k=3) resulted in the best overlap between clusters and phenotypes, regardless of the number of principal components (FIG. 3B, 12B, 12C) and exhibited a good Silhouette profile (FIG. 3C). Based on the overlap between clusters and phenotypes, normoglycemic, prediabetes and diabetes-like cluster labels were assigned (FIG. 3D). While it was noted that the diabetes-like cluster included more participants with T2D compared to the other clusters, and the normoglycemic-like cluster included more participants with normoglycemia, the prediabetes-like included more participants with prediabetes compared to the other clusters, but also almost half of participants with normoglycemia (43%, FIG. 3D).

To investigate which proteomics features are associated with the clusters, proteins that might be already altered in some, potentially undiagnosed, participants with normoglycemia were of particular interest. Within normoglycemic participants, differential expression was performed of plasma proteins between participants assigned to the normoglycemic-like and diabetes-like clusters. Out of the DE proteins identified above, 28 proteins were identified, most of which were over-expressed in plasma samples of participants with normoglycemia assigned to the diabetes-like cluster (FDR<=0.01, |coefficient|>=0.15, FIG. 3E). Many of these proteins are involved in immune response, which might signal higher inflammation in a subset of normoglycemic participants.

Clusters are Also Associated with Differences in Physical Performance and Echocardiogram

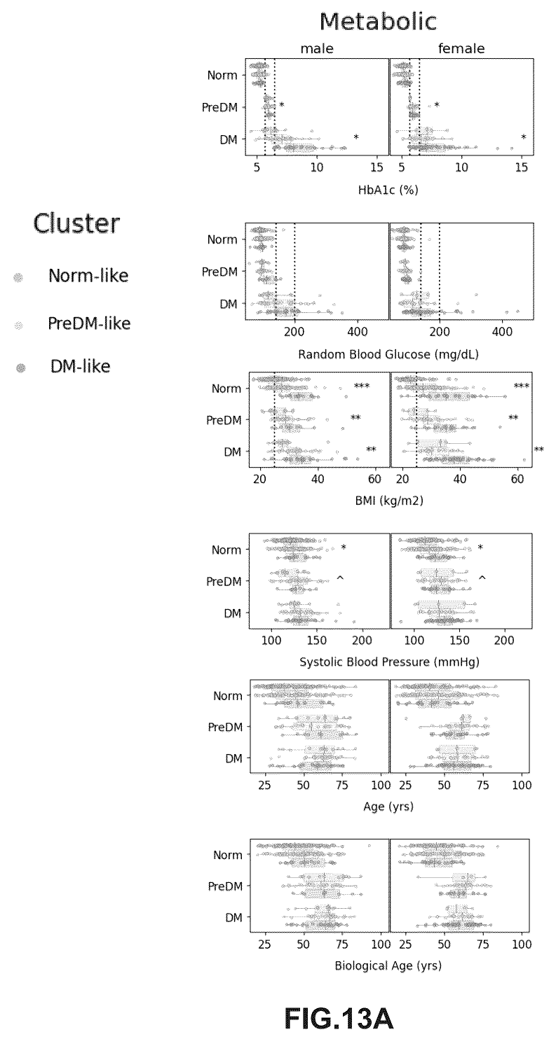

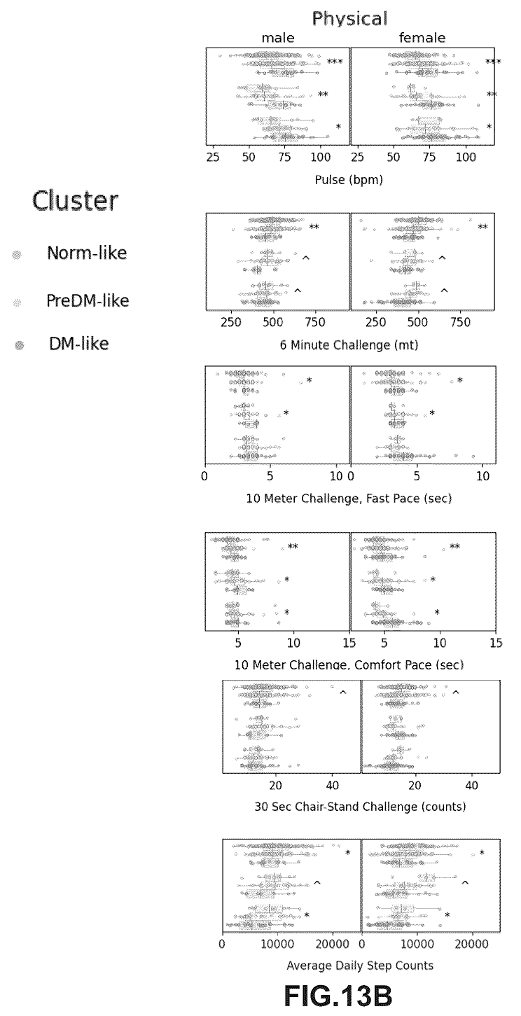

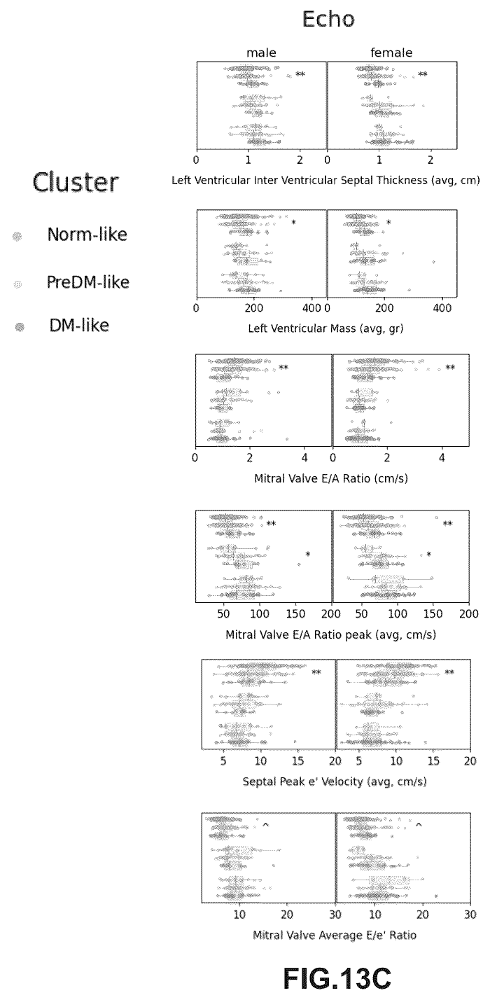

To help demonstrate the relevance of the clusters, differences between the cluster groups were examined at the metabolic, physical performance and cardiac-health level within each phenotype. The distribution of metabolic, physical performance and cardiac features across phenotypes and clusters were examined for each sex, although tests for statistical significance were performed considering the two sexes together because of limited sample size (FIGS. 4A-4C, 13A-13C). Of these, biological age, predicted from DNA methylation assay, physical performance features, excluding pulse rate, and echocardiogram-derived features were not part of the clustering input features, thus representing an orthogonal validation to the cluster assignment.

Several metabolic features were significantly different across cluster groups. HbA1c had clinically minor, but statistically significant, differences between cluster groups for all phenotypes, especially within the T2D phenotype, suggesting that the proteins used to assign cluster groups may provide some additional value for further differentiating diabetes status (FIGS. 4A, 13A). Highly significant differences in BMI and systolic blood pressure were observed between participants with normoglycemia assigned to the normoglycemic-like and diabetes-like clusters (FIG. 4A). A non-significant trend was appreciated in both actual and methylation age within the group of patients with normoglycemia, where participants in the diabetes-like subgroup were older than in the normoglycemic-like subgroup (FIG. 4A).

Features associated with physical performance were also significantly different between participants classified as diabetes-like and those normoglycemic-like, especially within the normoglycemic group (FIG. 4B). Since pulse rate was the only one of the physical performance features used for clustering, this finding suggests that the additional clinical and molecular markers of diabetes identified might also be related to physical performance overall. The average daily step count from the Verily Study Watch, in particular, was significantly higher for participants with T2D in the normoglycemic-like subgroup compared to those in the diabetes-like subgroup (FIG. 4B).

Finally, since diabetes is often associated with cardiovascular comorbidities (Ma et al. 2022), the distribution of features derived from echocardiogram images was compared between cluster groups for each phenotype. Measurements related to left ventricular size and mitral valve blood flow were focused on, since alterations in these have been previously reported in patients with diabetes (Palmieri et al. 2001)(Methods). Indeed, left ventricular mass and left ventricular septal thickness were significantly higher in participants in the diabetes-like normoglycemic subgroup compared to the normoglycemic-like normoglycemic subgroup (FIG. 4C). This could be a sign of ventricular hypertrophy, which is associated with hypertension (Aronow 2017) and is common in clinically diagnosed T2D patients (Mohan et al. 2021). Measurements of mitral valve blood flow were significantly different in participants in the diabetes-like normoglycemic subgroup compared to the normoglycemic-like subgroup: mitral valve E/A ratio, septal peak e′ velocity and mitral valve E/e′ ratio were lower and mitral valve E/A ratio peak was higher (FIG. 4C).

### Clinical and Proteomics Features Combined Best Predict Diabetes Status

Having observed significant differences at the clinical and molecular level between participants with T2D and normoglycemia, the ability of different feature sets were compared to differentiate T2D from normoglycemia without using HbA1c or blood glucose (these were initially used to refine the clinical diabetes phenotype and might lead to inflated performance of any model). Three models were built using three sets of features: clinical features only, proteomics features only and clinical and proteomics features combined (FIG. 14A, Methods). A ridge-logistic regression model was trained on participants with normoglycemia (n=467, 77%) and T2D (n=155, 23%) with clinical and proteomics data to predict diabetes status in a repeated cross-validation setting for model selection for interpretation (FIG. 14B, Methods), and evaluated the performance using repeated nested cross-validation on the same dataset (Krstajic et al. 2014) (FIG. 14C, Methods). To address the high data dimensionality, feature selection was performed as a preprocessing step inside the cross-validation pipelines for the proteomics only and combined datasets, with the number of selected features being a hyperparameter to tune (Methods). Clinical features were already filtered by excluding highly correlated features as described (Methods section).

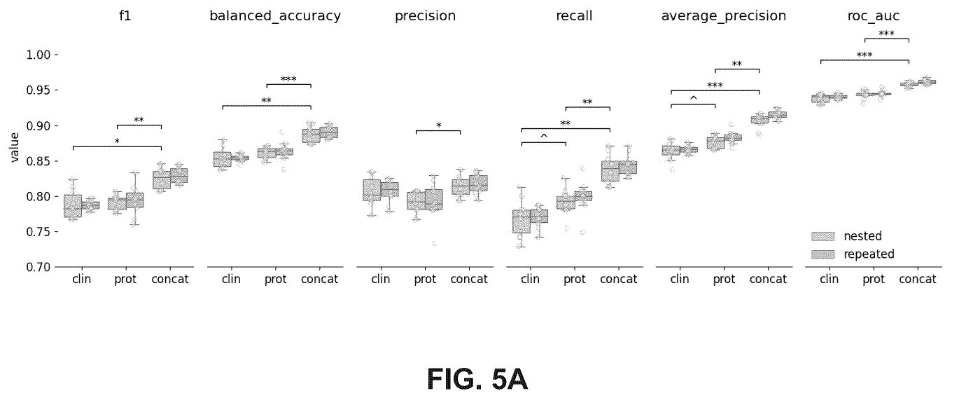

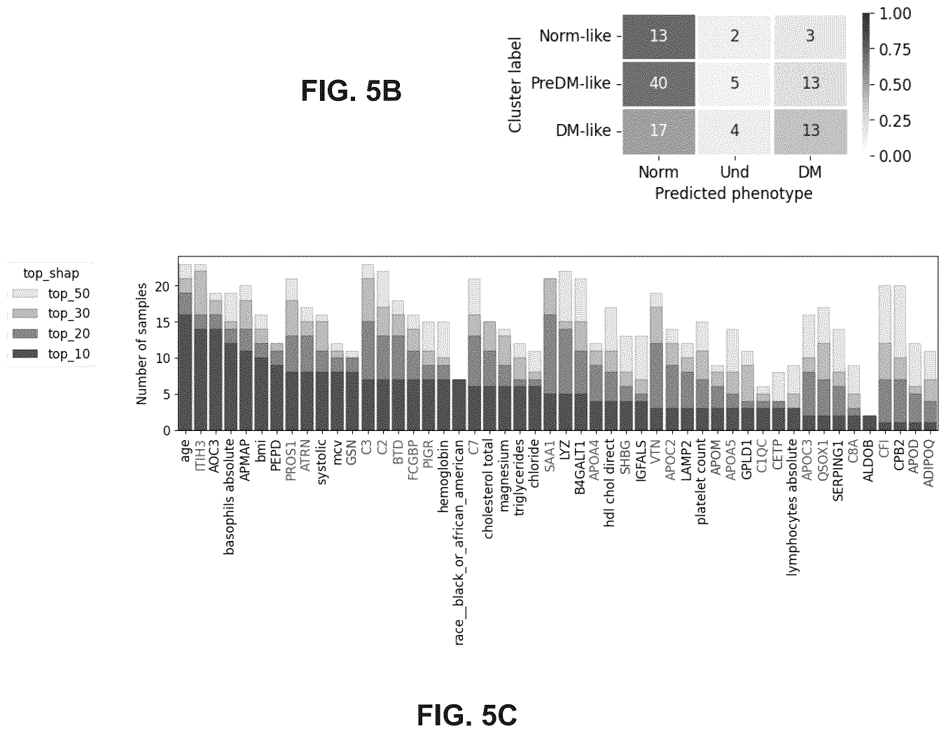

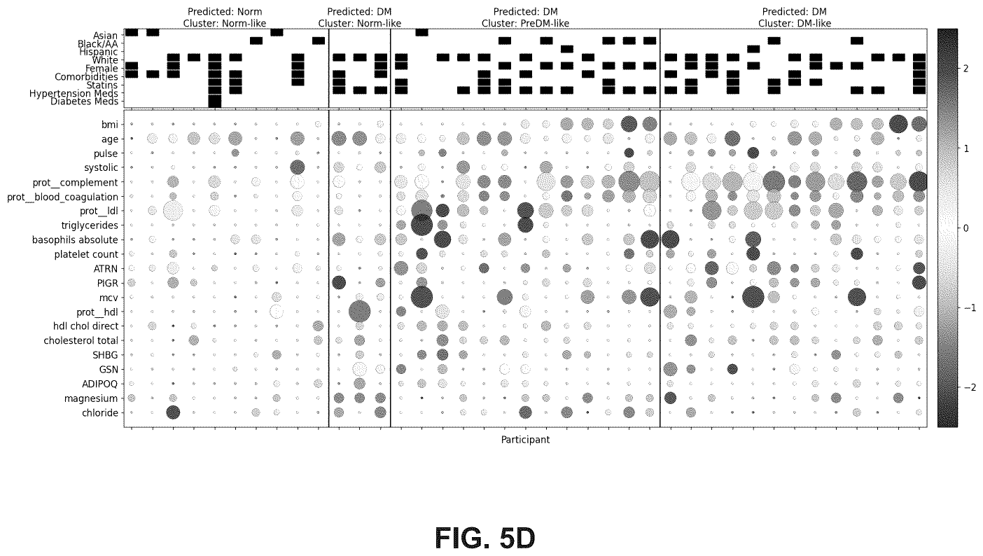

Model performance was compared between the datasets by testing the differences across several performance metrics within the repeated nested cross-validation setting (FIG. 5A, Methods). The distribution of model performance across several performance metrics was similar between the metrics computed within the repeated cross-validation and the repeated nested cross-validation settings (FIG. 5A). The model using the combined dataset performed best consistently across all the performance metrics, except for precision (FIG. 5A). In this context, precision, also called positive predictive value, measures the proportion of patients actually clinically defined as ‘with T2D’ that are predicted ‘with T2D’ over the number of all participants predicted ‘with T2D.’ Therefore, lower precision would imply more individuals without T2D were predicted as having T2D. This is consistent with our hypothesis that incorporating additional protein biomarkers could refine current clinical diagnosis of T2D.

To understand the relationship between diabetes predictions and cluster assignment, and to inspect further which features are contributing to diabetes predictions at the individual level, the model selected with repeated cross-validation using the combined dataset to predict diabetes status for 110 participants with prediabetes throughout the study was applied. Of these, 29 (26%) were predicted ‘with T2D’ by the model with probability higher than 0.6, while 70 were predicted ‘with normoglycemia’ with probability lower than 0.4 (FIG. 5B). Most of the participants predicted ‘with T2D’ were assigned to the prediabetic-like or the diabetic-like clusters, with proportionally more participants assigned to the diabetic-like cluster, although not significantly (FIG. 5B).

To gain more insights into which features are contributing the most to predict diabetes status, the SHapley Additive exPlanations (SHAP) values (Lundberg and Lee 2017) were computed for all the features and counted how many times a feature has the highest ranking SHAP value across the 29 participants predicted with T2D (FIG. 5C).

To investigate feature contribution at the individual level the SHAP values (Lundberg and Lee 2017) were examined for the 27 participants with prediabetes predicted ‘with T2D’ and 10 participants with prediabetes predicted ‘with normoglycemia’ with the lowest prediction probability as control (FIG. 5D). Expectedly, known risk factors for diabetes, such as BMI, age and blood pressure, were positively contributing to diabetes predictions for some, but not all, individuals (FIG. 5D). Lipid measurements from a standard lipid panel were also associated with diabetes predictions. Specifically, triglycerides were positively contributing to diabetes predictions, while HDL and total cholesterol were negatively contributing to diabetes predictions. Similarly, lower magnesium and chloride were also found to contribute to diabetes predictions, with magnesium and chloride deficits in T2D patients having been reported before (Barbagallo and Dominguez 2015; Khan et al. 2019).

Leveraging the additive nature of SHAP values (Lundberg and Lee 2017), participant-level aggregated SHAP values were computed for groups of functionally related proteins (see Table 1 for the manually curated list of aggregated proteins). Consistently with the differential protein expression results, complement, coagulation and LDL transport-related proteins showed positive contribution to diabetes predictions in most participants with prediabetes, while HDL-related apolipoproteins showed negative contribution to diabetes predictions in some participants with prediabetes (FIG. 5D). Positive contributions to diabetes predictions were noted for individual proteins, such as attractin protein (ATRN), which is involved in immune cell signaling (Duke-Cohan et al. 1998), and PIGR, which is involved in inflammatory response and hepatic malignancy (Sphyris and Mani 2011), and negative contributions for other individual proteins such as sex hormone-binding globulin (SHBG), whose levels have been shown to be inversely associated with diabetes risk (Aroda et al. 2020) and adiponectin (ADIPOQ), also inversely associated with diabetes risk as well as obesity and insulin resistance (Achari and Jain 2017).

Finally, while the same proteomics and clinical features were associated with T2D across multiple participants, examining SHAP values at the participant level highlighted how the contribution of each feature to diabetes prediction can vary between individuals. For example, qualitatively inspecting FIG. 5D, it was observed that some people are predicted ‘with T2D’ because they have high BMI and inflammatory markers, whereas others are older and have higher triglycerides, or others yet have high inflammatory markers and lipid dysregulation.

### DISCUSSION

This example has identified differential plasma proteomic profiles for T2D and prediabetes states, which could enable a more refined stratification of individuals at risk or living with the disease beyond what is possible using merely clinical information. Functionally and based on expression patterns, the proteins in these profiles are consistent with known features of T2D pathophysiology. Moreover, the combination of these profiles with clinical features allowed the development of a logistic regression model that could predict future type-2 diabetic disease status with accuracy. Our clustering/model also identified normoglycemic participants and participants with prediabetes that exhibit metabolic, physical and cardiovascular features that resemble T2D, suggesting that our approach may be useful for further patient stratification and risk management.

This type of analysis was enabled by the availability of a unique research resource such as the PBHS cohort, consisting of deeply phenotyped individuals, both healthy and spanning multiple disease areas, including diabetes. The collection of multi-modal data ranging from clinical, to digital and molecular profiling allows for an integrative characterization of diseases. This is particularly true for complex conditions, like T2D, which present individual phenotypic differences.

As part of PBHS, one of the largest proteomics datasets was generated, profiling almost one thousand individuals with a range of dysglycemia, including participants with diabetes, prediabetes and normoglycemic. Comparing plasma proteins in people with diabetes and normoglycemic revealed that inflammatory and blood coagulation markers are overexpressed in people with diabetes. This is consistent with the emerging role of systemic inflammation in the pathophysiology of T2D and associated metabolic disorders, which has generated increasing interest in inflammation as a target for intervention (Tsalamandris 2019).

In particular, it was found that proteins of the complement system are overexpressed in people with diabetes. The complement system, originally viewed as a supportive first line of defense against microbial invaders, is increasingly being studied for its role in the initiation and progression of metabolic disorders including obesity, insulin resistance and T2D (Shim et al. 2020). Many individuals with T2D in the PBHS cohort were overweight or obese, which contributes to the overexpression of inflammatory markers in plasma, but it was found that some complement proteins, including component 3 (C3), complement factor B (CFB) and complement factor I (CFI), were also overexpressed in participants with T2D and normal weight. The liver (mainly hepatocytes) is responsible for biosynthesis of about 80-90% of plasma complement components (Qin and Gao 2006). It was found that, anatomically, most of the DE proteins in this example were liver-centric, a finding largely consistent with results of previous transcriptional analyses of micro-dissected liver tissue that reported overexpression of immune-related genes in the zone closer to the central vein (McEnerney et al. 2017) and pronounced zonation of active complement gene transcription, specifically in periportal and interzonal hepatocytes (Andrews et al. 2022). Yet, some genes that were detected in liver biopsies from GTEx were not detected in the single cell dataset, for example APOC2 and APOC4, both of which respond to metabolic cues in the liver by activation of transcription factors and nuclear hormone receptors (Wolska et al. 2017). This may be due to these genes being expressed below the detection limit in single cells. Another explanation could be that these genes are detected in only some GTEx samples from donors with pre-existing conditions, such as diabetes.

Additionally, it was found that proteins involved in blood coagulation and hemostasis were also overexpressed in the plasma of participants with T2D. Examples of these proteins included fibrinogen subunits alpha (FGA), beta (FGB) and gamma (FGG), plasminogen (PLG) and plasmin inhibitor (SERPINF2) (Kattula, Byrnes, and Wolberg 2017). Overexpression of hemostatic proteins in conjunction with overexpression of inflammatory markers could represent a response to endothelial cell damage in blood vessels, as the metabolic burden of T2DM, including insulin resistance, hyperglycemia and release of excess free fatty acids, along with other metabolic abnormalities affects vascular wall by a series of events including endothelial dysfunction, platelet hyperactivity, oxidative stress and low-grade inflammation (Kaur, Kaur, and Singh 2018). Indeed, it has been suggested that T2D and/or other cardiometabolic diseases can each cause reversible microvascular injury with accompanying dysfunction, which in time may or may not become irreversible and anatomically identifiable disease (Kaze et al. 2021; Horton and Barrett 2021).

Altogether, the physiological observations related to the DE proteins suggest that the liver zone close to the central vein might be related to immune response and to overall inflammation, based on the signals from complement genes. Additional multi-omics studies can help elucidate the interrelation between T2D and liver dysfunction, particularly nonalcoholic fatty liver disease (NAFLD) including steatohepatitis (NASH) (Gastaldelli and Cusi 2019; Tanase et al. 2020); and how they might be linked through inflammatory mechanisms such as complement activation (Guo et al. 2022).

Clustering analysis of participants with normoglycemia, diabetes and prediabetes based on clinical and proteomics features showed that 10% of normoglycemic participants had a clinico-molecular profile that resembled that of participants with T2D. At the proteomics level, these participants, mostly overweight and obese, consistently showed elevated levels of inflammatory and blood coagulation proteins. This suggests that measuring the presence of inflammatory and hemostatic pathways in plasma might help stratify within groups with seemingly similar levels of glycemic control. Participants such as these, normoglycemic by clinical standards but stratified closer to those with T2D, might be at high risk for diabetes, supporting the need for a holistic phenotypic assessment to properly diagnose diabetes or general metabolic dysregulation. Furthermore, normoglycemic participants in the diabetes-like cluster had, on average, poorer physical performance than the other normoglycemic participants and altered echocardiogram readouts indicative of left ventricular hypertrophy, which may be also linked to hypertension. Somewhat conversely, the findings regarding physical activity levels recorded via wearable device indicated that participants with T2D in the normoglycemic-like subgroup, that is, with lower inflammatory markers, were more physically active.

In addition, several of our echocardiographic-related observations are consistent with prior reports establishing a relationship between echocardiographic abnormalities and T2D, particularly, abnormalities related to left ventricular size and mitral valve blood flow (ref).

Finally, a machine learning model was trained to predict diabetes status based on clinical and proteomics features and applied it to participants with prediabetes. The model trained on both clinical and proteomics features combined, performed better than the models trained on clinical or proteomics features alone, achieving over 85% balanced accuracy. This performance is consistent with other clinical and/or molecular diabetes classifiers. To investigate the contribution of each feature to the model classification, the model was applied to participants with prediabetes and examined the SHAP values, which quantify how much a feature is contributing to diabetes classification for each individual. Consistent with the rest of the analysis, many participants with prediabetes who were predicted as ‘with T2D’ by the model showed elevated levels of complement and hemostatic proteins. However, differences in feature contribution between individuals could also be appreciated, emphasizing the importance of assessing metabolic disorders in a holistic and personalized manner.

As described herein, a large scale longitudinal clinical cohort of deeply phenotyped participants across a health spectrum can be the source for integrative analyses that explore multiple layers of a complex disease. This holistic approach examines clinical and molecular features for each patient. In this case, provided herein is a deep molecular characterization of the T2D continuum at the individual level, identified differential proteomic profiles in individuals with normoglycemia, prediabetes and T2D consistent with known pathophysiologic features of the disease. These profiles can serve as areas for disease targeting and also as complementary information to better stratify patients and tailor personalized interventions.

### REFERENCES

Achari, Arunkumar E., and Sushil K. Jain. 2017. “Adiponectin, a Therapeutic Target for Obesity, Diabetes, and Endothelial Dysfunction.” International Journal of Molecular Sciences 18 (6): 1321.
Adusumilli, Ravali, and Parag Mallick. 2017. “Data Conversion with ProteoWizard msConvert.” Proteomics, 339-68.
Andrews, Tallulah S., Jawairia Atif, Jeff C. Liu, Catia T. Perciani, Xue-Zhong Ma, Cornelia Thoeni, Michal Slyper, et al. 2022. “Single-Cell, Single-Nucleus, and Spatial RNA Sequencing of the Human Liver Identifies Cholangiocyte and Mesenchymal Heterogeneity.” Hepatology Communications 6 (4): 821-40.
Arges, Kristine, Themistocles Assimes, Vikram Bajaj, Suresh Balu, Mustafa R. Bashir, Laura Beskow, Rosalia Blanco, et al. 2020. “The Project Baseline Health Study: A Step towards a Broader Mission to Map Human Health.” Npj Digital Medicine 3 (1): 1-10.
Aroda, Vanita R., Costas A. Christophi, Sharon L. Edelstein, Leigh Perreault, Catherine Kim, Sherita H. Golden, Edward Horton, and Kieren J. Mather. 2020. “Circulating Sex Hormone Binding Globulin Levels Are Modified with Intensive Lifestyle Intervention, but Their Changes Did Not Independently Predict Diabetes Risk in the Diabetes Prevention Program.” BMJ Open Diabetes Research and Care 8 (2): e001841.
Barbagallo, Mario, and Ligia J. Dominguez. 2015. “Magnesium and Type 2 Diabetes.” World Journal of Diabetes 6 (10): 1152-57.
Behdenna, Abdelkader, Julien Haziza, Chloé-Agathe Azencott, and Akpéli Nordor. 2020. “pyComBat, a Python Tool for Batch Effects Correction in High-Throughput Molecular Data Using Empirical Bayes Methods.” bioRxiv. https://doi.org/10.1101/2020.03.17.995431.
Benjamini, Yoav, and Yosef Hochberg. 1995. “Controlling the False Discovery Rate: A Practical and Powerful Approach to Multiple Testing.” Journal of the Royal Statistical Society. Series B, Statistical Methodology 57 (1): 289-300.
Blondel, Vincent D., Jean-Loup Guillaume, Renaud Lambiotte, and Etienne Lefebvre. 2008. “Fast Unfolding of Communities in Large Networks.” Journal of Statistical Mechanics 2008 (10): P10008.
Calínski, T., and J. Harabasz. 2007. “A Dendrite Method for Cluster Analysis.” Communications in Statistics—Theory and Methods, June. https://www.tandfonline.com/doi/abs/10.1080/03610927408827101.
Cauwenberghs N, Haddad F, Daubert M A, et al. Clinical and echocardiographic diversity associated with physical fitness in the Project Baseline Health Study: implications for heart failure staging. J Card Fail. 2023;:51071-9164(23)00149-5
CDC. 2023. “Diabetes Tests.” Centers for Disease Control and Prevention. Mar. 1, 2023. https://www.cdc.gov/diabetes/basics/getting-tested.html.
Cessie, S. Le, and J. C. Van Houwelingen. 1992. “Ridge Estimators in Logistic Regression.” Journal of the Royal Statistical Society. Series C, Applied Statistics 41 (1): 191.
Chatterjee, Ranee, Lydia Coulter Kwee, Neha Pagidipati, Lynne H. Koweek, Priyatham S. Mettu, Francois Haddad, David J. Maron, et al. 2022. “Multi-Dimensional Characterization of Prediabetes in the Project Baseline Health Study.” Cardiovascular Diabetology 21 (1): 1-13.
Davies, D. L., and D. W. Bouldin. 1979. “A Cluster Separation Measure.” IEEE Transactions on Pattern Analysis and Machine Intelligence 1 (2): 224-27.
Demichev, Vadim, Christoph B. Messner, Spyros I. Vemardis, Kathryn S. Lilley, and Markus Ralser. 2020. “DIA-NN: Neural Networks and Interference Correction Enable Deep Proteome Coverage in High Throughput.” Nature Methods 17 (1): 41-44.
Deutsch, Eric W., Gilbert S. Omenn, Zhi Sun, Michal Maes, Maria Pernemalm, Krishnan K. Palaniappan, Natasha Letunica, et al. 2021. “Advances and Utility of the Human Plasma Proteome.” Journal of Proteome Research 20 (12): 5241-63.
Duke-Cohan, J. S., J. Gu, D. F. McLaughlin, Y. Xu, G. J. Freeman, and S. F. Schlossman. 1998. “Attractin (DPPT-L), a Member of the CUB Family of Cell Adhesion and Guidance Proteins, Is Secreted by Activated Human T Lymphocytes and Modulates Immune Cell Interactions.” Proceedings of the National Academy of Sciences of the United States of America 95 (19): 11336-41.
ElSayed N A, Aleppo G, Aroda V R, Bannuru R R, Brown F M, Bruemmer D, Collins B S, Cusi K, Hilliard M E, Isaacs D, Johnson E L. 4. Comprehensive medical evaluation and assessment of comorbidities: Standards of Care in Diabetes—2023. Diabetes Care. 2023 Jan. 1; 46(Supplement_1):s49-67.
Farbstein, Dan, and Andrew P. Levy. 2014. “HDL Dysfunction in Diabetes: Causes and Possible Treatments.” Expert Review of Cardiovascular Therapy, January. https://doi.org/10.1586/erc.11.182.
Feingold, Kenneth R. 2021. “Introduction to Lipids and Lipoproteins.” In Endotext, edited by Kenneth R. Feingold, Bradley Anawalt, Marc R. Blackman, Alison Boyce, George Chrousos, Emiliano Corpas, Wouter W. de Herder, et al. South Dartmouth (MA): MDText.com, Inc.
Gastaldelli, Amalia, and Kenneth Cusi. 2019. “From NASH to Diabetes and from Diabetes to NASH: Mechanisms and Treatment Options.” JHEP Reports 1 (4): 312.
Geyer, Philipp E., Eugenia Voytik, Peter V. Treit, Sophia Doll, Alisa Kleinhempel, Lili Niu, Johannes B. Müller, et al. 2019. “Plasma Proteome Profiling to Detect and Avoid Sample-Related Biases in Biomarker Studies.” EMBO Molecular Medicine 11 (11): e10427.
Guo, Zhenya, Xiude Fan, Jianni Yao, Stephen Tomlinson, Guandou Yuan, and Songqing He. 2022. “The Role of Complement in Nonalcoholic Fatty Liver Disease.” Frontiers in Immunology 13 (September): 1017467.
Hagberg, Aric A., Daniel A. Schult, and Pieter J. Swart. 2008. “Exploring Network Structure, Dynamics, and Function Using NetworkX.” In Proceedings of the 7th Python in Science Conference, edited by Gaēl Varoquaux, Travis Vaught, and Jarrod Millman, 11-15. Pasadena, CA USA.
Horton, William B., and Eugene J. Barrett. 2021. “Microvascular Dysfunction in Diabetes Mellitus and Cardiometabolic Disease.” Endocrine Reviews 42 (1): 29.
Horvath, Steve. 2013. “DNA Methylation Age of Human Tissues and Cell Types.” Genome Biology 14 (10): 1-20.
“Inflammation in Obesity, Diabetes, and Related Disorders.” 2022. Immunity 55 (1): 31-55.
Johnson, W. Evan, Cheng Li, and Ariel Rabinovic. 2007. “Adjusting Batch Effects in Microarray Expression Data Using Empirical Bayes Methods.” Biostatistics 8 (1): 118-27.
Kattula, Sravya, James R. Byrnes, and Alisa S. Wolberg. 2017. “Fibrinogen and Fibrin in Hemostasis and Thrombosis.” Arteriosclerosis, Thrombosis, and Vascular Biology 37 (3): e13.
Kaur, Raminderjit, Manpreet Kaur, and Jatinder Singh. 2018. “Endothelial Dysfunction and Platelet Hyperactivity in Type 2 Diabetes Mellitus: Molecular Insights and Therapeutic Strategies.” Cardiovascular Diabetology 17. https://doi.org/10.1186/s12933-018-0763-3.
Kaze, Arnaud D., Prasanna Santhanam, Sebhat Erqou, Rexford S. Ahima, Alain Bertoni, and Justin B. Echouffo-Tcheugui. 2021. “Microvascular Disease and Incident Heart Failure Among Individuals With Type 2 Diabetes Mellitus.” Journal of the American Heart Association, June. https://doi.org/10.1161/JAHA.120.018998.
Khan, Rashid Naseem, Farhana Saba, Syedhh Fatima Kausar, and Mohammad Hassan Siddique. 2019. “Pattern of Electrolyte Imbalance in Type 2 Diabetes Patients: Experience from a Tertiary Care Hospital:” Pakistan Journal of Medical Sciences Quarterly 35 (3). https://doi.org/10.12669/pjms.35.3.844.
Krstajic, Damjan, Ljubomir J. Buturovic, David E. Leahy, and Simon Thomas. 2014. “Cross-Validation Pitfalls When Selecting and Assessing Regression and Classification Models.” Journal of Cheminformatics 6 (1): 10.
Lundberg, Scott M., and Su-In Lee. 2017. “A Unified Approach to Interpreting Model Predictions.” In Advances in Neural Information Processing Systems, edited by I. Guyon, U. Von Luxburg, S. Bengio, H. Wallach, R. Fergus, S. Vishwanathan, and R. Garnett. Vol. 30. Curran Associates, Inc. https://proceedings.neurips.cc/paper_files/paper/2017/file/8a20a8621978632d76c43dfd28b67767-Paper.pdf.
McEnerney, Laura, Kara Duncan, Bo-Ram Bang, Sandra Elmasry, Meng Li, Toshio Miki, Sadeesh K. Ramakrishnan, Yatrik M. Shah, and Takeshi Saito. 2017. “Dual Modulation of Human Hepatic Zonation via Canonical and Non-Canonical Wnt Pathways.” Experimental & Molecular Medicine 49 (12): e413.
Mering, Christian von, Lars J. Jensen, Berend Snel, Sean D. Hooper, Markus Krupp, Mathilde Foglierini, Nelly Jouffre, Martijn A. Huynen, and Peer Bork. 2005. “STRING: Known and Predicted Protein-protein Associations, Integrated and Transferred across Organisms.” Nucleic Acids Research 33 (suppl_1): D433-37.
Owora, Arthur H. 2018. “Commentary: Diagnostic Validity and Clinical Utility of HbA1c Tests for Type 2 Diabetes Mellitus.” Current Diabetes Reviews 14 (2): 196-99.
Pedregosa, F., G. Varoquaux, A. Gramfort, V. Michel, B. Thirion, O. Grisel, M. Blondel, et al. n.d. “Scikit-Learn: Machine Learning in PYthon.” Journal of Machine Learning Research: JMLR.
“Prevalence of Both Diagnosed and Undiagnosed Diabetes.” 2022. Sep. 21, 2022. https://www.cdc.gov/diabetes/data/statistics-report/diagnosed-undiagnosed-diabetes.html.
Qin, X., and B. Gao. 2006. “The Complement System in Liver Diseases.” Cellular & Molecular Immunology 3 (5). https://pubmed.ncbi.nlm.nih.gov/17092430/.
Rhee, Eun-Jung, Kyungdo Han, Seung-Hyun Ko, Kyung-Soo Ko, and Won-Young Lee. 2017. “Increased Risk for Diabetes Development in Subjects with Large Variation in Total Cholesterol Levels in 2,827,950 Koreans: A Nationwide Population-Based Study.” PloS One 12 (5): e0176615.
Shim, Kyumin, Rayhana Begum, Catherine Yang, and Hongbin Wang. 2020. “Complement Activation in Obesity, Insulin Resistance, and Type 2 Diabetes Mellitus.” World Journal of Diabetes 11 (1): 1-12.
“Silhouettes: A Graphical Aid to the Interpretation and Validation of Cluster Analysis.” 1987. Journal of Computational and Applied Mathematics 20 (November): 53-65.
Sphyris, Nathalie, and Sendurai A. Mani. 2011. “pIgR: Frenemy of Inflammation, EMT, and HCC Progression.” Journal of the National Cancer Institute 103 (22): 1644-45.
Szklarczyk, Damian, Annika L. Gable, David Lyon, Alexander Junge, Stefan Wyder, Jaime Huerta-Cepas, Milan Simonovic, et al. 2018. “STRING v11: Protein-protein Association Networks with Increased Coverage, Supporting Functional Discovery in Genome-Wide Experimental Datasets.” Nucleic Acids Research 47 (D1): D607-13.
Tanase, D. M., E. M. Gosav, C. F. Costea, M. Ciocoiu, C. M. Lacatusu, M. A. Maranduca, A. Ouatu, and M. Floria. 2020. “The Intricate Relationship between Type 2 Diabetes Mellitus (T2DM), Insulin Resistance (IR), and Nonalcoholic Fatty Liver Disease (NAFLD).” Journal of Diabetes Research 2020 (July). https://doi.org/10.1155/2020/3920196.
F“The Synthesis and Secretion of Plasma Proteins in the Liver.” 1978. Pathology 10 (4): 394.
Tomah, Shaheen, Naim Alkhouri, and Osama Hamdy. 2020. “Nonalcoholic Fatty Liver Disease and Type 2 Diabetes: Where Do Diabetologists Stand?” Clinical Diabetes and Endocrinology 6 (June): 9.
Tsalamandris, Sotirios. 2019. “The Role of Inflammation in Diabetes: Current Concepts and Future Perspectives,” April. https://assets.radcliffecardiology.com/s3fs-public/article-pdf/2020-12/ECR_Tousoulis_WEB.pdf.
Vinh, Nguyen Xuan, Julien Epps, and James Bailey. 2010. “Information Theoretic Measures for Clusterings Comparison: Variants, Properties, Normalization and Correction for Chance.” Journal of Machine Learning Research: JMLR 11 (95): 2837-54.
Wolska, Anna, Richard L. Dunbar, Lita A. Freeman, Masako Ueda, Marcelo J. Amar, Denis O. Sviridov, and Alan T. Remaley. 2017. “Apolipoprotein C-IL: New Findings Related to Genetics, Biochemistry, and Role in Triglyceride Metabolism.” Atherosclerosis 267 (December): 49-60.
Yang, Robert Y., Jie Quan, Reza Sodaei, Francois Aguet, Ayellet V. Segre, John A. Allen, Thomas A. Lanz, et al. 2018. “A Systematic Survey of Human Tissue-Specific Gene Expression and Splicing Reveals New Opportunities for Therapeutic Target Identification and Evaluation.” bioRxiv. https://doi.org/10.1101/311563.

What is claimed is:

1. A method for determining a type-2 diabetes status in a subject, comprising:
(a) obtaining a biological sample from the subject;
(b) determining the level of one or more proteins selected from the group consisting of electron transfer flavoprotein dehydrogenase (ETFDH), albumin (ALB), keratin 81, 83, 86 (KRT81;KRT83;KRT86), paraoxonase 1 (PON1), paraoxonase 3 (PON3), adiponectin, C1Q and collagen domain containing (ADIPOQ), sex hormone binding globulin (SHBG), apolipoprotein D (APOD), apolipoprotein A1 (APOA1), apolipoprotein M (APOM), cholesteryl ester transfer protein (CETP), cartilage acidic protein 1 (CRTAC1), GLI pathogenesis-related 2 (GLIPR2), cadherin 13 (CDH13), C-type lectin domain family 3 member B (CLEC3B), gelsolin (GSN), complement C7 (C7), fibroblast activation protein alpha (FAP), collectin subfamily member 10 (COLEC10), collectin subfamily member 11 (COLEC11), heat shock protein family A (Hsp70) member 5 (HSPA5), heat shock protein family A (Hsp70) member 8 (HSPA8), fc gamma binding protein (FCGBP), colony stimulating factor 1 receptor (CSF1R), quiescin sulfhydryl oxidase 1 (QSOX1), fumarylacetoacetate hydrolase (FAH), galectin 3 binding protein (LGALS3BP), polymeric immunoglobulin receptor (PIGR), apolipoprotein A5 (APOA5), cathepsin D (CTSD), serpin family D member 1 (SERPIND1), haptoglobin (HP), haptoglobin-related protein (HPR), serum amyloid A1 (SAA1), S100 calcium binding protein A8 (S100A8), S100 calcium binding protein A9 (S100A9), procollagen C-endopeptidase enhancer (PCOLCE), fibrinogen gamma chain (FGG), fibrinogen alpha chain (FGA), fibrinogen beta chain (FGB), complement C8 alpha chain (C8A), complement C8 gamma chain (C8G), complement C6 (C6), complement C9 (C9), inter-alpha-trypsin inhibitor heavy chain 3 (ITIH3), gamma-glutamyl hydrolase (GGH), C-reactive protein (CRP), lipopolysaccharide binding protein (LBP), complement C2 (C2), mannosidase alpha class 1A member 1 (MAN1A1), apolipoprotein C4 (APOC4), apolipoprotein C2 (APOC2), apolipoprotein C3 (APOC3), apolipoprotein A4 (APOA4), apolipoprotein H (APOH), alpha-1-microglobulin/bikunin precursor (AMBP), serpin family F member 1 (SERPINF1), complement Clq B chain (C1QB), complement Clq C chain (C1QC), complement Clr subcomponent like (C1RL), complement Clr (C1R), complement C1s (C1S), serpin family A member 10 (SERPINA10), coagulation factor XI (F11), protein C, inactivator of coagulation factors Va and VIIIa (PROC), serpin family F member 2 (SERPINF2), complement factor properdin (CFP), biotinidase (BTD), butyrylcholinesterase (BCHE), afamin (AFM), attractin (ATRN), complement factor H (CFH), complement factor H related 2 (CFHR2), complement C3 (C3), complement factor H (CFH), complement factor B (CFB), complement factor I (CFI), kininogen 1 (KNG1), vitronectin (VTN), complement C5 (C5), hemopexin (HPX), coagulation factor X (F10), orosomucoid 2 (ORM2), complement component 4 binding protein alpha (C4BPA), protein S (PROS1), proteoglycan 4 (PRG4), amyloid P component, serum (APCS), and coagulation factor IX (F9); and
(c) transforming the level of one or more proteins into a probability score, wherein an increase in the probability score indicates an increased likelihood of a type-2 diabetes status.

2. The method of claim 1, wherein the one or more proteins are selected from the group consisting of CDH13, CETP, CLEC3B, CRTAC1, GSN, SHBG, C3, CFB, and VTN.

3. The method of claim 1, wherein the one or more proteins are selected from the group consisting of AMBP, ALB, APOA1, HP, SAA1, APOC3, HPX, APOH, VTN, ORM2, APCS, APOA5, FGG, FGB, FGA, CRP, ITIH3, KNG1, SERPINF2, C3, C6, CFH, C5, C8A, AFM, C4BPA, C9, LBP, CFI, PON1, F11, PROC, F9, APOM, CFB, C2, SERPIND1, SERPINA10, F10, PRG4, BCHE, PON3, APOA4, SHBG, APOC2, COLEC10, APOC4, C8G, PIGR, and COLEC11.

4. The method of claim 1, wherein the one or more proteins are selected from the group consisting of APOA1, APOM, SAA1, CFI, C5, and F11.

5. The method of claim 1, wherein the one or more proteins are selected from the group consisting of SHBG, FAH, LGALS3BP, PIGR, GGH, C1RL, MAN1A1, CRP, LBP, C9, FGA, FGG, SAA1, S100A8, S100A9, SERPIND1, HP, HP;HPR, APOC4, ORM2, CFH;CFHR2, C3, CFH, CFB, CFI, PRG4, APCS, and F9.

6. The method of claim 1, wherein the one or more proteins are selected from the group consisting of ITIH3, PROS1, ATRN, C3, C2, BTD, FCGBP, PIGR, C7, SAA1, APOA4, VTN, APOC2, APOA5, C1QC, APOC3, QSOX1, C8A, CFI, GSN, SHBG, APOM, CETP, APOD, ADIPOQ.

7. The method of claim 1, wherein the one or more proteins are selected from the group consisting of ITIH3, PROS1, ATRN, C3, C2, BTD, FCGBP, PIGR, C7, SAA1, APOA4, VTN, APOC2, APOA5, C1QC, APOC3, QSOX1, C8A, and CFI.

8. The method of claim 1, wherein the one or more proteins are selected from the group consisting of GSN, SHBG, APOM, CETP, APOD, and ADIPOQ.

9. The method of claim 1, wherein the one or more proteins are selected from the group consisting of SHBG, APOD, C3, VTN, C2, GSN, CFB, CFH, APOA1, CFH;CFHR2, CFI, QSOX1, ADIPOQ, HSPA5;HSPA8, C4BPA, ATRN, PON3, CETP, PIGR, SERPIND1, PROS1, FGA, C7, APOC4, FGB, FGG, C1RL, BTD, LGALS3BP, F9. HPX, CDH13, GGH, CTSD, SERPINF1, CLEC3B, HP;HPR, NKG1, CRP, CRTAC1, COLEC10, LBP, C5, PCOLCE, AFM, C1QB, KRT81;KRT83;KRT86, APOC3, ETFDH, C6, BCHE, APOM, HP, PRG4, C8G, SERPINA10, APOC2, SERPINF2, ALB, APCS, COLEC1I, FCGBP, and F11.

10. The method of claim 1, wherein the one or more proteins are selected from the group consisting of CFP, CFB, CFH, C3, C9, C8G, C8A, C5, C7, S100A9, S100A8, LBP, FGB, FGA, APCS, CSF1R, C6, CFI, C4BPA, C2, C1S, C1RL, C1R, C1QB, C1QC.

11. The method of claim 1, wherein the one or more proteins are selected from the group consisting of PROS1, FGB, F9, C7, C5CFI, VTN, CFB, C4BPA, CETP, APOD, APOC1, APOM, APOA1, APOH, APOC4, APOC3, APOC2, APOA4, and LCAT.

12. The method of claim 1, wherein the subject is a subject having or suspected of having type 2 diabetes.

13. The method of claim 1, wherein the biological sample is whole blood, plasma, serum, stool, saliva, tears, urine, or one or more facial swabs.

14. The method of claim 1, wherein the biological sample is whole blood or urine.

15. The method of claim 1, wherein the biological sample is not urine.

16. The method of claim 1, wherein step (c) includes applying a statistical model to the determined levels of the one or more proteins to assign a probability score for the subject.

17. The method of claim 1, wherein the method further comprises reviewing one or more clinical features of the subject to assign the probability score to the subject.

18. The method of claim 17, wherein step (c) further comprises integrating the one or more clinical features of the subject with the determined levels of the one or more proteins of the sample before or after the transforming to obtain a probability score.

19. The method of claim 17, wherein the one or more clinical features comprise:
sex at birth, racial identity, age, one or more respiratory rate measurements, one or more triglyceride measurements, one or more waist circumference measurements, one or more glycated hemoglobin (HbA1c) measurements, one or more blood glucose measurements, one or more fasting blood glucose measurements, hypercholesterolemia status, hypertension status, one or more oral glucose tolerance test (OGTT) results, one or more total cholesterol measurements, one or more low-density lipoprotein (LDL) measurements, one or more high-density lipoprotein (HDL) measurements, one or more weight measurements, one or more body mass index (BMI) calculations, one or more blood pressure (BP) measurements, one or more pulse rate measurements, one or more average step count measurements, one or more methylation age measurements, one or more echocardiogram images, one or more ventricular mass measurements, one or more ventricular septal measurements, one or more mitral valve blood flow measurements, or any combination of any thereof.

20. The method of claim 17, wherein the one or more clinical features comprise age, sex, comorbidity status, hypertension medication status, statin status, diabetes medication status, or any combination of any thereof.

21. The method of claim 17, wherein the one or more clinical features comprise:
sex at birth, one or more HbA1c % measurements, one or more random glucose measurements, one or more BMI measurements, one or more systolic BP measurements, age, biological age, one or more pulse measurements, one or more 6 minute challenge measurements, one or more 10 meter challenge (fast pace) measurements, one or more 10 meter challenge (comfort pace) measurements, one or more 30 second stair stand challenge measurements, average daily step counts, one or more left ventricular inter ventricular septal thickness measurements, one or more left ventricular mass measurements, one or more mitral valve E/A ratio measurements, one or more mitral valve E/A ratio peak measurements, one or more septal peak e′ velocity measurements, or any combination of any thereof.

22. The method of claim 17, wherein the one or more clinical features of the subject comprise:
age, race, one or more absolute basophil measurements, one or more BMI measurements, one or more systolic BP measurements, one or more mcv measurements, one or more hemoglobin measurements, one or more total cholesterol measurements, one or more magnesium measurements, one or more triglyceride measurements, one or more chloride measurements, one or more hdl cholesterol direct measurements, one or more platelet count measurements, one or more absolute lymphocyte measurements, or any combination of any thereof.

23. The method of claim 17, wherein the one or more clinical features of the subject comprise:
one or more BMI measurements, age, one or more pulse measurements, one or more systolic BP measurements, one or more aggregated complement protein measurements, one or more aggregated blood coagulation protein measurements, one or more LDL measurements, one or more triglyceride measurements, one or more absolute basophil measurements, one or more platelet count measurements, one or more mcv measurements, one or more HDL measurements, one or more total cholesterol measurements, one or more magnesium measurements, one or more chloride measurements, or any combination of any thereof.

24. The method of claim 17, wherein the one or more clinical features of the subject comprise:
sex, age, race, smoking status, comorbidity status, statin usage status, hypertension medication usage status, or any combination of any thereof.

25. The method of claim 17, wherein the one or more clinical features comprise:
mean corpuscular hemoglobin concentration (MCHC), mean corpuscular hemoglobin (MCH), MCV, bilirubin direct, bilirubin total, HDL direct, vitamin D, carbon dioxide, magnesium, reaction pH, eosinophils, eosinophils absolute, basophils, basophils absolute, lactic dehydrogenase, alanine aminotransferase (ALAT), aspartate aminotransferase (ASAT), albumin urine, albumin/creatine ratio, enzymatic creatinine serum, urea nitrogen, chloride, sodium, potassium, t-4 (thyroxine) free, phosphorus (inorganic), mean platelet volume (MPV), thyroid stimulating hormone, red cell count, hematocrit, hemoglobin, total cholesterol, LDL, total serum protein, albumin, calcium, lymphocytes, volgens Modification of Diet in Renal Disease (MDRD) glomerular filtration rate (GFR), absolute lymphocytes, platelet count, creatinine random ur, specific gravity, reticulocytes %, reticulocytes absolute, glucose, HbA1c, bmi, waist circumference, alkaline phosphatase, gamma-glutamyl transferase, respiratory rate, c-reactive protein (CRP) high sensitivity, pulse, diastolic (BP), systolic (BP), triglycerides, uric acid, neutrophil segmentation, total neutrophils, white cell count, absolute neutrophils, total neutrophils absolute, or any combination of any thereof.

26. The method of claim 17, wherein the one or more clinical features comprise:
BMI, triglycerides, pulse, absolute lymphocytes, absolute basophiles, platelet count, CRP high sensitivity, MDRD GFR, ALAT, red cell count, absolute neutrophils, absolute monocytes, absolute reticulocytes, absolute eosinophils, calcium, systolic bp, respiratory rate, potassium, ASAT, diastolic (BP), eric acid, protein total serum, thyroid stimulating hormone, MPV, enzymatic creatinine serum, MCHC, total cholesterol, albumin, hemoglobin, vitamin D, sodium, chloride, magnesium, HDL cholesterol direct, mcv, or any combination of any thereof.

27. The method of claim 17, wherein the one or more clinical features are not blood cell percentages, waist circumference, calculated LDL, or hematocrit.

28. The method of claim 17, wherein the one or more clinical features comprise one or more blood glucose measurements, one or more glycated hemoglobin (HbA1c) measurements, or both.

29. The method of claim 17, wherein the clinical features are not HbA1c or blood glucose measurements.

30. The method of claim 17, wherein the one or more clinical features comprise:
one or more HbA1c measurements, one or more BMI measurements, one or more systolic BP measurements, one or more glucose measurements, one or more physical performance measurements, or any combination of any thereof.

31. The method of claim 17, wherein the one or more clinical features comprise:
one or more left ventricular size measurements, one or more left ventricular mass measurements, one or more left ventricular septal thickness measurements, one or more mitral valve blood flow measurements, one or more mitral valve E/A ratio measurements, one or more septal peak e′ velocity measurements, one or more mitral valve E/e′ ratio measurements, one or more mitral valve E/A ratio peak measurements, or any combination of any thereof.

32. The method of claim 17, wherein the one or more clinical features comprise:
one or more BMI measurements, age, one or more blood pressure measurements, one or more triglyceride measurements, one or more magnesium measurements, one or more chloride measurements, or any combination of any thereof.

33. The method of claim 1, further comprising prescribing or administering one or more therapeutic treatments to the subject if the probability score indicates a likelihood of type-2 diabetes status.

34. The method of claim 1, further comprising changing one or more therapeutic treatments for the subject if the probability score indicates a likelihood of type-2 diabetes status.

35. The method of claim 1, wherein the level of the one or more proteins is determined using an assay selected from the group consisting of an enzyme-linked immunosorbent assay, a flow cytometry analysis, a dot blot assay, a Western blot assay, sequencing, liquid chromatography mass spectrometry (LCMS), orbitrap mass spectrometry, and an immunohistochemical localization assay.

36. The method of claim 1, wherein the method includes detecting 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, or 87 of the proteins.

37. The method of claim 1, wherein the method includes detecting 2 or more of the proteins.

38. The method of claim 1, wherein the method includes detecting all of the proteins.

39. The method of claim 33, wherein the administering reduces one or more symptoms of type 2 diabetes.

40. The method of claim 39, wherein the one or more symptoms of type 2 diabetes comprises hyperglycemia, fatigue, blurry vision, weight loss, excessive urination, excessive and persistent thirst, slow healing cuts or wounds.

41. The method of claim 1, wherein the subject is receiving a therapeutic agent and the method further comprises changing a dose or therapeutic agent given to the subject.

42. The method of claim 41, wherein the method further comprises increasing the dose of the therapeutic agent given to the subject.

43. The method of claim 33, wherein the method further comprises prescribing or administering an additional therapeutic agent to the subject.

44. The method of claim 1, wherein the type-2 diabetes status is prediabetes.
